SEQUENCE LISTING (30 sequences)

SEQ ID NO: 1 - DNA, 45 bp, Artificial Sequence
  primer
ctgccccgacgataggcttaatcgtggtctggtgaagttctacgg

SEQ ID NO: 2 - DNA, 41 bp, Artificial Sequence
  primer
ccagtgcgacctgctgggtgggtattgttcgccgccagtac

SEQ ID NO: 3 - DNA, 20 bp, Artificial Sequence
  primer
gatcaccgatttcaccaacc

SEQ ID NO: 4 - DNA, 22 bp, Artificial Sequence
  primer
cttttgagatcagcaacgtcag

SEQ ID NO: 5 - DNA, 16 bp, Artificial Sequence
  primer
tgcgccatgtcccgct

SEQ ID NO: 6 - DNA, 19 bp, Artificial Sequence
  primer
tgagttaacccacctgacg

SEQ ID NO: 7 - DNA, 25 bp, Artificial Sequence
  meditator
tccgcagcaagtgggctctacgacc

SEQ ID NO: 8 - DNA, 41 bp, Artificial Sequence
  Erster Teil Mediatorsonde
ggtcgtagagcccacttgctgcggatgcgccatgtcccgct

SEQ ID NO: 9 - DNA, 46 bp, Artificial Sequence
  Nachweismolekül
gaccggccaagacgcgccggtctgttggtcgtagagcccagaacga

SEQ ID NO: 10 - DNA, 47 bp, Artificial Sequence
  primer
cagcttcctcattgatggtttctttttaacaccatgctaaacacagt

SEQ ID NO: 11 - DNA, 45 bp, Artificial Sequence
  primer
tgttgcaccaggccagataattttgtactggtagttcctgctatg

SEQ ID NO: 12 - DNA, 19 bp, Artificial Sequence
  primer
attatcagaaggagccacc

SEQ ID NO: 13 - DNA, 21 bp, Artificial Sequence
  primer
catcctatttgttcctgaagg

SEQ ID NO: 14 - DNA, 25 bp, Artificial Sequence
  primer
tttaacatttgcatggctgcttgat

SEQ ID NO: 15 - DNA, 19 bp, Artificial Sequence
  primer
gagatccaaggggaagtga

SEQ ID NO: 16 - DNA, 30 bp, Artificial Sequence
  Mediator
ccatgcctcaggagctcagttcggtcagtg

SEQ ID NO: 17 - DNA, 55 bp, Artificial Sequence
  Erster Teil Mediatorsonde
cactgaccgaactgagctcctgaggcatggtttaacatttgcatggctgcttgat

SEQ ID NO: 18 - DNA, 45 bp, Artificial Sequence
  Nachweismolekül
caccggccaagacgcgccggtgtgttcactgaccgaactggagca

SEQ ID NO: 19 - DNA, 21 bp, Artificial Sequence
  primer
gtgaaggtcggtgtgaacgga

SEQ ID NO: 20 - DNA, 20 bp, Artificial Sequence
  primer
ttctgccgatgcccccatgt

SEQ ID NO: 21 - DNA, 20 bp, Artificial Sequence
  primer
gcatcctgcaccaccaactg

SEQ ID NO: 22 - DNA, 21 bp, Artificial Sequence
  primer
ggtttcttactccttggaggc

SEQ ID NO: 23 - DNA, 21 bp, Artificial Sequence
  primer
cagatccacgacggacacatt

SEQ ID NO: 24 - DNA, 20 bp, Artificial Sequence
  primer
gagcttcccgttcagctctg

SEQ ID NO: 25 - DNA, 40 bp, Artificial Sequence
  Mediator
taaagccatagccgtactagctgctccagttcggtcagtg

SEQ ID NO: 26 - DNA, 60 bp, Artificial Sequence
  Erster Teil einer Meditorsonde
cactgaccgaactggagcagctagtacggctatggctttagcatcctgcaccaccaactg

SEQ ID NO: 27 - DNA, 45 bp, Artificial Sequence; identical to SEQ ID NO: 18
  Nachweismolekül

SEQ ID NO: 28 - DNA, 20 bp, Artificial Sequence
  Mediator
tcgttctgggctctacgacc

SEQ ID NO: 29 - DNA, 36 bp, Artificial Sequence
  Erster Teil einer Mediatorsonde
ggtcgtagagcccagaacgatgcgccatgtcccgct

SEQ ID NO: 30 - DNA, 30 bp, Artificial Sequence
  Nachweismolekül
ttttttttttggtcgtagagcccagaacga

(19) United States
(12) Patent Application Publication
(10) Pub. No.: US 2019/0376126 A1
(43) Pub. Date: Dec. 12, 2019

(54) TWO-PART MEDIATOR PROBE

(71) Applicants: ALBERT-LUDWIGS-UNIVERSITÄT FREIBURG, Freiburg (DE); HAHN-SCHICKARD-GESELLSCHAFT FÜR ANGEWANDTE FORSCHUNG E.V., Villingen-Schwenningen (DE)

(72) Inventors: Martin TROTTER, Freiburg (DE); Simon WADLE, Waldshut-Tiengen (DE); Felix VON STETTEN, Freiburg (DE); Lisa BECHERER, Lahr-Sulz (DE)

(21) Appl. No.: 16/472,322

(22) PCT Filed: Dec. 15, 2017
(86) PCT No.: PCT/EP2017/083039

§ 371 Date: Jun. 21, 2019

(30) Foreign Application Priority Data
Dec. 23, 2016 (DE) 10 2016 125 592.0
Dec. 23, 2016 (DE) 10 2016 125 597.1
Feb. 17, 2017 (DE) 10 2017 103 284.3

(51) Int. Cl. C12Q 1/6823; C12Q 1/6832; C12Q 1/6853; C12Q 1/686

(52) U.S. Cl. CPC C12Q 1/6823; C12Q 1/6832; C12Q 1/6853; C12Q 1/686; C12Q 2537/137; C12Q 2525/161; C12Q 2525/205; C12Q 2525/301; C12Q 2561/101

(57) ABSTRACT

The present invention concerns a mediator probe for the detection of at least one target molecule comprising at least two oligonucleotides. A first oligonucleotide of the mediator probe according to the invention comprises a probe region and a mediator binding region, wherein the probe region has an affinity to a target molecule and/or template molecule, and the mediator binding region has an affinity to at least one mediator. At least one further oligonucleotide of the mediator probe is a mediator which is bound to the first oligonucleotide of the mediator probe via the mediator binding region and has an affinity for at least one detection molecule, wherein the mediator triggers a detectable signal by interaction with the detection molecule after release from the first oligonucleotide of the mediator probe. Furthermore, the present invention concerns a system comprising at least one mediator probe according to the invention and at least one detection molecule, as well as a method for the detection of at least one target molecule.

### INTRODUCTION

The present invention concerns a mediator probe comprising at least two oligonucleotides for the detection of at least one target molecule. A first oligonucleotide of the mediator probe according to the invention comprises a probe region and a mediator binding region, wherein the probe region has an affinity to a target molecule and/or template molecule, and the mediator binding region has an affinity to at least one mediator. At least one further oligonucleotide of the mediator probe is a mediator, which is bound to the first oligonucleotide of the mediator probe via the mediator binding region and has an affinity for at least one detection molecule, wherein the mediator triggers a detectable signal by interaction with the detection molecule after release from the first oligonucleotide of the mediator probe. Furthermore, the present invention concerns a system comprising at least one mediator probe according to the invention and at least one detection molecule, as well as a method for the detection of at least one target molecule.

### State-of-the-Art

DNA amplifications are used, among other things, in clinical diagnostics for the investigation of diseases. DNA amplification involves making a large number of copies of the desired target sequence so that an initially small amount of DNA can be made visible.

DNA amplification can be performed by various methods. In addition to PCR, which requires thermal cycling between about 60° C. and 95° C., isothermal amplification methods such as LAMP (62° C.) or RPA (39° C.) are also used. Various approaches are available for real-time tracking of DNA amplification and detection of amplification products. Bioluminescence, chemoluminescence, turbidity measurements and fluorescence-based detection methods enable, among other things, the detection and quantification of the DNA to be examined. Most of the above methods are only capable of detecting the total amount of amplified DNA in the sample and cannot distinguish between different target sequences. These methods are therefore only suitable for so-called singleplex verifications. Fluorescence-based, as well as luminescence, electrochemical and other detection methods open up further application possibilities. In addition to intercalating detection molecules, which interact unspecifically with DNA strands, modified oligonucleotides are used. The latter can be used for target sequence-specific analyses, while intercalating detection molecules often lead to a false-positive detection of non-specific by-products.

In clinical analytics and in vitro diagnostics, it makes sense to be able to detect several target molecules within a reaction in parallel, since, for example, different bacteria or viruses can be the cause of various diseases. Accordingly, multi-analyte verifications are of great importance, for which some examples are listed below: For example, not only the ABO genotyping is relevant for the blood group determination but also the generation of the Human Neutrophil Antigen Profile (HNA), which has to be determined for blood and tissue transfusions. Parallel testing of blood donor samples for HIV variants and hepatitis B or C viruses is also routinely performed using immunoassays or nucleic acid-based techniques. The specific detection of pathogens requires the determination of several genomic loci in order to allow a derived diagnosis after short analysis times.

The activity determination of different marker and control genes allows the creation of an expression profile. This can be used, for example, to identify oncogenes that influence cell division and cell differentiation and are therefore closely correlated to cancer, or to make predictions about the efficacy of certain drugs depending on the patient's genotype (personalized medicine). Also frequently represented hereditary diseases can be detected in molecular biological (prenatal) diagnostics, including cystic fibrosis (cystic fibrosis), phenylketonuria (metabolic disorder) and thalassemia (degradation of erythrocytes). Furthermore, the joint detection of inflammation markers such as procalcitonin or cytokines allows conclusions to be drawn about the severity of an infection.

If, as in the examples above, the diagnostic question requires the analysis of several target molecules, genetic loci or other markers as well as internal controls or references, methods that only allow the determination of a single parameter per analysis are usually of little significance. If, on the other hand, different individual analyses are carried out in parallel to record several parameters, this is uneconomical: The sample solution must be divided into several reaction batches in which different target molecules are detected. A problem that arises is as follows: by dividing the sample solutions into n aliquots, the amount of substance in the individual reaction is reduced by a factor of 1/n, whereby the sensitivity of the detection reaction is reduced accordingly.

In order to avoid these disadvantages, homogeneous or heterogeneous reaction approaches are developed to capture several parameters, in which different target molecules are detected in parallel. Various oligonucleotides labeled for detection are used, which bind specifically to the target molecule to be detected. In the direct dependence between labeled oligonucleotide and target molecule described above, the problem arises that the use of a new probe is necessary if a new experimental question arises, e.g. if a different genotype of a virus is to be detected. This makes it necessary to develop new, labeled oligonucleotides for detection for each new experimental question. This is time-consuming and expensive due to the modifications of the oligonucleotides required for the detection.

As an alternative to the parallel detection of different target sequences in homogeneous reaction approaches, oligonucleotides can be immobilized for detection on a solid phase (heterogeneous detection). Depending on the signaling position at the fixed phase, the presence of certain target sequences can be inferred. The direct dependence between the labeled oligonucleotide and the target molecule again leads to the problem that the immobilized oligonucleotides have to be adapted to the experimental problem. For each new experimental question, new oligonucleotides have to be immobilized on a solid phase. This is very time-consuming due to the complex manufacturing process.

The disadvantageous use of target sequence-specific oligonucleotides with labels for detection or at different positions of a solid phase leads to the necessity of a universal detection method, which is sequence-specific and nevertheless cost-effective. In a universal method, the sequence of signal-generating oligonucleotides (detection molecules) is independent of the target sequence to be detected. The same optimized signal generating oligonucleotides can be used for different target sequences. As a result, working time and thus labor costs can be saved, since the signal-generating oligonucleotides do not have to be readjusted for each detection reaction.

Sequence-specific, universal detection methods are already known, but they have some disadvantages. In particular, enzymes are used that are only compatible with certain amplification methods, mostly non-isothermal amplification methods. These include, for example, the multi-analyte reporter system according to Faltin et al. 2012 and the use of universal duplex probes according to Yang et al. 2008. In the methods mentioned, an enzyme, for example a polymerase, with nuclease activity is absolutely necessary, although the polymerases used in LAMP or RPA do not possess this nuclease activity. So-called beach displacement polymerases are hardly used for sequence-specific, universal detection. In addition, the procedure according to Yang et al. 2008 runs the risk of generating false-positive signals.

WO 2013079307 A1 describes a universally applicable method for the detection of at least one target molecule using a system comprising a mediator probe and a universal reporter molecule. A mediator release by cleavage requires an enzyme with nuclease activity. In a PCR, the polymerase has this nuclease activity in most forms, which is why no additional enzymes are required for this amplification method. However, in isothermal amplification methods, such as LAMP or RPA, or in PCDR, the polymerases used do not possess this nuclease activity. Consequently, mediator release by cleavage is only possible through the addition of enzymes that exhibit nuclease activity. A disadvantage of this is that additional enzymes interact with other components in the reaction mix and can thus influence the efficiency of the detection reaction. In addition, the need for additional enzymes increases the cost of the reaction mix for the detection reaction. In addition, the use of additional enzymes results in an additional workload for optimizing the detection reaction under the changed conditions.

US 2016/0312271 A1 describes a universally applicable method for the detection of at least one target molecule using a system comprising a cleavable probe and a universal detection molecule. In the procedure according to US 2016/0312271 A1, the detection reaction is triggered analogous to WO 2013079307 A1 by cleavage of an oligonucleotide. Accordingly, the same disadvantage occurs that enzymes with nuclease activity are necessary.

State-of-the-art procedures are also described in which primers are used, comprising a hairpin formation sequence, covalently bound fluorophores or bound fluorescence-labeled probes. In addition, a second, fluorescence-labeled probe is used, which binds to the amplicon and can interact with the first fluorophore. The target sequence-specific hairpin formation sequence and the target sequence-specific second probe lead to the disadvantage that the fluorophores are not attached to universal sequence sections and therefore this method cannot be used universally. The signal generation must therefore be optimized separately for new detection reactions. In addition, the additional second probe poses a risk when strand displacement polymerases are used, as a first probe bound to the primer can be extended and thus displace the second probe.

At this point, the sequence-specific detection of amplification products using Strand displacement polymerases in a LAMP according to Tanner et al. 2012 should be mentioned. With this detection method, the fluorescence donor and fluorescence acceptor are bound to target sequence-specific oligonucleotides, which is why this method is not universal and therefore has the disadvantage that detection must be optimized for each new detection reaction.

Furthermore, detection methods on the basis of molecular beacons were described, which contain primer sequences and thus have target sequence-specific regions. Another disadvantage results from the dependence on the target sequence because the signal-generating labels are located on target sequence-specific oligonucleotides, which is why this detection method is not universally applicable. In addition, the fluorescence yield and the balance between closed and open conformation of the molecular beacon depend on the primer sequence. The detection reaction must therefore be optimized separately for each new detection reaction.

The universal technologies described in the literature that use strand displacement polymerases also have some disadvantages. For example, using a molecular beacon hybridized to a primer according to Li et al. 2006 or CN 101328498 A, there is a risk that a false-positive signal will be generated in the absence of the target molecule due to the stability of the hairpin structure of the molecular beacon (Li et al. 2007). In addition, this detection method has so far only been used for amplification reactions via PCR. The function of isothermal amplification methods has not been proven and the stability of the hairpin structure, which is even more pronounced at lower temperatures (LAMP, RPA), speaks against the use of this method in combination with isothermal amplification. The use of universal, fluorescence-labeled primers according to G. J. Nuovo et al. 1999 in turn involves the risk that hybridization of the universal primer can also lead to false-positive signal generation in non-specific amplification products.

None of the state-of-the-art methods allows the parallel detection of different molecules and molecule classes, such as proteins and nucleic acids, in a single step, which could create a combined DNA-RNA-protein profile of a sample.

For diagnostic questions that require the analysis of several different target molecules from different substance classes, detection methods are advantageous that can detect different substance classes, such as proteins and nucleic acids, side by side. The detection methods described in the literature, which allow the simultaneous detection of several molecule classes, are either not universally applicable methods (Das et al. 2012) or have the additional disadvantage that the detection reaction has to be carried out in several stages (Linardy et al. 2016), which entails a great deal of work and time during implementation.

This results in the need for a sequence-specific, universal detection method that can simultaneously detect several analytes and circumvents the disadvantages of state-of-the-art methods. In addition, a detection method is required which can be used for different amplification methods, regardless of whether the latter are isothermal or non-isothermal.

The present invention is thus based on the task of providing a mediator probe as well as a system and method for the detection of at least one target molecule, which does not exhibit the disadvantages of the state-of-the-art described above. Accordingly, the task was to provide a mediator probe and a method, which essentially allows simultaneous, universal and/or sequence-specific detection of several analytes of different molecule classes.

### General Description of the Invention

The task is solved by the independent claims. Advantageous forms of execution result from the subclaims.

According to the invention, the present technical task is solved by providing a two-part mediator probe for the detection of at least one target molecule.

The invented mediator probe for the detection of at least one target molecule comprises at least two oligonucleotides and is characterized in that
a) a first oligonucleotide comprises a probe region and a mediator binding region, wherein
   the probe region has an affinity to a target molecule and/or template molecule, and
   the mediator binding region has an affinity for at least one mediator, and
b) at least one further oligonucleotide is a mediator which is
   is bound via the mediator binding region to the first oligonucleotide of the mediator probe, and
   has an affinity for at least one detection molecule, wherein the mediator triggers a detectable signal after release from the first oligonucleotide of the mediator probe by interaction with the detection molecule.

A mediator probe according to the invention thus comprises a first molecule or oligonucleotide comprising a mediator binding region and a probe region, and a second molecule or oligonucleotide, the mediator. The probe region of the first molecule has an affinity to the target and/or template molecule and the mediator binding region has an affinity to the mediator or mediators. A template molecule is used if the probe region cannot interact directly with the target molecule. Consequently, a template molecule serves as a mediator between the target molecule and the probe region.

After the binding of the probe region to a target molecule and/or template molecule, the mediator is displaced from the mediator binding region by a molecule, preferably an enzyme with DNA strand separating effect, and in certain versions with additional polymerizing effect, preferably a beach displacement polymerase. Interaction of the mediator with a detection molecule triggers a detectable signal. A strand displacement polymerase has a strand displacement activity and displaces the strand complementary to the amplified strand during amplification.

It was completely surprising that by astutely taking advantage of the universal applicability of the mediator probe, it was possible to use it for the detection of different target molecules. Surprisingly, it is possible to detect several target molecules simultaneously in one sample using several mediator probes according to the invention.

The mediator probe according to the invention enables universal sequence-dependent detection of any nucleic acid sequences of the target molecule and/or template molecule. A detection molecule can be used which has a fixed design independent of the target sequence or probe region of the mediator probe.

Surprisingly, it is possible that the release of the mediator and the subsequent signal generation by interaction with a detection molecule can be applied to different amplification methods and are not limited to specific systems. By astutely taking advantage of the respective conditions in different amplification methods, the above-mentioned mediator release can easily be adapted to the respective system.

There are various state-of-the-art systems for the detection of target nucleic acid sequences based on labeled oligonucleotide probes or primers. However, in contrast to the method according to the invention as described here, which is based on the system according to the invention and the mediator probe according to the invention, these methods are not universal detection methods that can be performed with different target specific molecules independently of the target sequence.

Signal-generating modifications, such as fluorophore and quencher, are often applied to target sequence-specific oligonucleotides (primers). In these cases, the signal-generating molecule cannot be used for different detection reactions because it has to be individually designed and optimized for each target molecule. A great advantage of the present invention over the state-of-the-art is therefore the universal applicability of the signal-generating universal detection molecules called in connection with the mediator probe according to the invention. These universal reporter molecules contain signal-generating molecules, but no target sequence-specific segments. The universal reporter molecules can be used to detect different target molecules without having to redesign or optimize the reporter molecules. Only the two-part mediator probe has to be adapted. The mediator probe according to the invention also features a simplified primer design. In contrast to state-of-the-art systems, the oligonucleotides used as primers do not have to contain molecules capable of fluorescence or linker molecules, nor do they contain a second target sequence-specific region.

During the amplification process, the fluorophore- and quencher-labeled remainder of the primer is displaced from the target molecule by the strand-dispersing polymerase, thus restoring the signal to its original state and not guaranteeing a sustained signal change. In addition, many state-of-the-art detection methods require an enzyme with nuclease activity, e.g. a polymerase, but in the case of LAMP or RPA, the polymerases used in this invention do not possess this nuclease activity. According to the invention, nuclease activity is preferably not necessary, which is why isothermal amplification methods are also used. Many state-of-the-art detection methods, however, cannot be used with the isothermal amplification method, such as LAMP.

In contrast to state-of-the-art procedures, the procedure according to the invention preferably does not split the mediator probe. In contrast to the described system, the mediator probe is two-part, so that the release of the mediator can take place without splitting a covalent bond by displacement. This allows advantageously the real-time detection of a target molecule in an isothermal amplification reaction where no polymerase with nuclease activity is used. In addition, by using multiple mediators per mediator probe, the detection signal can be amplified and the different mediators of a probe can generate different detection signals. According to the invention, only one specific binding site to the target molecule is necessary even if several mediators are used per mediator probe. For known state-of-the-art systems or procedures, however, several complete mediator probe systems must be used if several mediators are to be released.

State-of-the-art systems are known that contain primers with a target molecule-specific sequence and can hybridize with a fluorophore-labeled probe. In contrast to these methods, however, the mediator of the two-part mediator probe according to the invention preferably does not carry any markings for signal generation. If a fluorophore is bound to a primer by hybridization through a separate probe, the fluorophore may still be influenced by the target sequence-specific portion of the primer. If guanine bases are present in the primer sequence, the fluorescence yield of the fluorophore can be negatively influenced by the guanine bases. Thus, an influence/dependency of the fluorescence yield of the fluorophore on a separate probe by target sequence-specific sections in the primer sequence arises. In addition, in most cases no detection molecule with a universal sequence is used.

State-of-the-art primers are also described with a hairpin formation sequence and a corridorophore bound covalently or by a hybridized probe. The hairpin formation sequence contains a second target sequence-specific region. Preferably, however, the first part of the mediator probe according to the invention contains only one target sequence-specific region or sequence. In addition, the mediator probe according to the invention preferably does not contain a hairpin formation sequence and only one target sequence-specific region. In addition, known systems require a primer and often two additional labeled probes, whereby at least one probe is target sequence specific. Such primer/probe systems thus consist of two or three molecules with target molecule-specific sequences. In contrast, the invented mediator probe preferably only has one molecule with a target molecule-specific sequence.

In addition, the mediator probe in certain versions does not contain a marking. The mediator also preferably does not contain a target molecule-specific sequence.

The invention at hand is a completely new and surprising development in view of the known state-of-the-art. The state-of-the-art does not reveal similar detection systems that work with strand-dispersing activity of enzymes. Rather, it describes detection methods that can be carried out under PCR conditions with polymerases that have no strand displacing activity.

It is completely surprising that a mediator probe, a system and a procedure have been developed according to the invention to take advantage of the active displacement of the mediator by an enzyme with a strand-dispersing effect. Many state-of-the-art processes are based on the nuclease activity of the polymerases used, which is absolutely necessary. There is no obvious connection between the fission and displacement reaction. In addition, it is not obvious or trivial for an expert to adapt known methods that use the nuclease activity of polymerases in such a way that they function with displacing enzymes, since a large number of reaction conditions have to be modified or recombined in different ways.

It is also not obvious for an expert to combine known universal detection methods with known methods using hybridized target sequence-specific primers and target sequence-specific probes without applying a universal detection principle.

The advantages of the mediator probe according to the invention described here apply in particular to the preferred versions of execution of the mediator probe, the system according to the invention and the procedure according to the invention.

Detection of a target molecule in the context of the invention means that the presence of the target molecule in the sample to be investigated is detected quantitatively or qualitatively. According to the invention, a target molecule is a molecule whose presence is to be detected in a sample. It is a biomolecule, such as, without limitation, a nucleic acid molecule, a protein, a peptide, a sugar molecule, a lipid, or combinations of these molecules, such as glycosylated proteins or other glycosylated biomolecules.

The term nucleic acids in the meaning of this invention includes, without limitation, DNA, RNA, PNA, ssDNA, dsDNA, RNA, mRNA, tRNA, IncRNA, ncRNA, microRNA, siRNA, rRNA, sgRNA, piRNA, rmRNA, snRNA, snoRNA, scaRNA, gRNA, viral RNA, or modified RNA such as LNA. Oligonucleotides in the sense of the present invention are nucleic acid molecules of relatively short length comprising approximately up to 200 nucleotides.

Oligonucleotides may be covalently and non-covalently linked to other molecules or chemical groups, such as fluorescence donors and/or fluorescence acceptors and block groups.

Sugar molecules within the meaning of this invention are in particular carbohydrates or saccharides and include mono-, di-, oligo- and polysaccharides. Glycosylation describes a series of enzymatic or chemical reactions in which carbohydrates are bound to proteins, lipids or other aglycones. The resulting reaction product is referred to as glycoside, in the case of proteins as glycoprotein or peptidoglycan, in the case of lipids as glycolipids.

In the sense of the invention, the term “lipids” refers to completely or at least largely water-insoluble (hydrophobic) substances, which, due to their low polarity, dissolve very well in hydrophobic (or lipophilic) solvents. Lipids are structural components in cell membranes and are also used in living organisms as energy stores or signal molecules. Most biological lipids are amphiphilic, i.e. they have a lipophilic hydrocarbon residue and a polar hydrophilic head group, which is why they form micelles or membranes in polar solvents such as water. The group of lipids includes in particular fatty acids, triacylglycerides (fats and fatty oils), waxes, phospholipids, sphingolipids, lipopolysaccharides and isoprenoids (steroids, carotenoids etc.).

The probe region is preferably complementary to a section of the target molecule and/or template molecule. The probe region of the first oligonucleotide of the mediator probe binds to a target molecule and/or template molecule. The binding takes place via the probe region of the mediator probe, as this has an affinity to the target molecule and/or template molecule. The mediator binding region does not need to have any affinity to the template molecule and does not need to have a complementary sequence segment.

The mediator preferably has a complementary area to a section of a detection molecule. The mediator binds to a detection molecule, triggering a detectable signal. The detectable signal allows conclusions to be drawn about the presence of the target molecule or template molecule. The template molecule itself can be the target molecule to be detected, or it can be associated with the target molecule so that information about the presence of the target molecule can be generated via the template molecule.

The detection molecule according to the invention is an oligonucleotide with which the target molecule can interact indirectly and may cause a detection reaction by processing (e.g. change of a fluorescence signal, electrochemical signal or mass).

A template molecule is a nucleic acid molecule that can be used if the reagents used for the invention detection method, e.g. primers or probes or the invention mediator probe, cannot interact directly with the target molecule. A template molecule can therefore be used as a mediator between target molecule and primer or probes. Aptamers are usually used as template molecules.

Aptamers are oligonucleotides, which, due to their structural properties, interact with or bind to other molecules or molecule complexes. The molecules bound by aptamers can be proteins, peptides, sugar molecules, lipid molecules, nucleic acid molecules or molecule complexes formed from these molecules. Aptamers can assume different conformations in the bound and unbound state, so that different sequence regions of an aptamer, for example, are accessible for interactions with complementary oligonucleotides such as primers or probes, depending on the conformation.

An interaction in the sense of this invention refers to the mutual interaction of different interacting molecules. This can be, for example, a covalent or non-covalent bond between two molecules, or an indirect bond mediated by one or more other molecules, for example within a molecular complex.

In the context of this invention, a detectable signal refers to any kind of change that can be measured physically or chemically. These changes include, without limitation, cleavage, digestion, strand duplication, internal hybridization, phosphorylation, dephosphorylation, amidation, binding or cleavage of a chemical group, fluorescence, phosphorescence or luminescence changes.

In a preferred design of the mediator probe according to the invention, the first oligonucleotide of the mediator probe and/or the mediator does not include a marker for signal generation. A decisive advantage of such an unlabeled mediator probe is that it can be used in a method according to the invention for the detection of at least one target molecule without the need for labor and costs for optimizing the new assay. In contrast to the state-of-the-art, the mediator probe consists of oligonucleotides, which can be synthesized cost-effectively without technically complex modifications such as fluorescence donors and/or fluorescence acceptors and block groups.

The term label refers preferentially to any atom or molecule which can be used to provide a detectable (preferably quantifiable) signal and which can be covalently or non-covalently bound to a nucleic acid or protein or other biomolecule. Labels can provide signals that can be detected by redox reactions, luminescence, fluorescence, radioactivity, colorimetry, gravimetry, X-ray diffraction or absorption, magnetism, enzymatic activity and the like.

In a preferred version, the first oligonucleotide of the mediator probe and/or the mediator contains one or more markers for signal generation, preferably a fluorescent molecule, a redox molecule, a luminescent molecule or another signal generating unit.

In another preferred version of the invention, the mediator contains one or more markers for signal generation, preferably a fluorescent molecule, a redox molecule, a luminescent molecule or another signal generating molecule or another signal generating unit. According to this invention, the mediator, after displacement from the mediator probe, can bind to a detection molecule, which also contains at least one label for signal generation, resulting in a detectable signal change.

For example, the mediator bound to the mediator probe can be marked with a fluorescence donor/acceptor which emits at a certain wavelength λ₁. After displacement of the mediator probe, the mediator can bind to a detection molecule labeled with a fluorescence acceptor/donor, which is emitted at a second wavelength λ₂, which is different to λ₁. The energy transfer from the fluorescence donor to the fluorescence acceptor via the FRET mechanism leads to a detectable increase in the radiation intensity of the fluorescence acceptor, which allows the emission of λ₂ to be detected. Alternatively, chemiluminescent or bioluminescent donor molecules can be used. Non-emissive fluorescence acceptors can also be used in preferred designs. By using detection molecules with different numbers of nucleotides, different target molecules can be distinguished simultaneously in one sample by means of a melting curve analysis.

By marking the mediator the universal character of the described detection method is not lost because the mediator is a universal, target sequence independent molecule. In further designs, several probes or primers per target molecule can be used to which labeled mediators are bound, whereby the sequences of the mediators and the labels may differ. For example, fluorescent dyes with different emission wavelengths can be used.

A preferred version of the mediator probe according to the invention is characterized in that the release of the mediator from the first oligonucleotide of the mediator probe occurs during amplification of the target molecule and/or template molecule to which the mediator probe is bound.

The term amplification or amplification reaction in the sense of this invention refers to the amplification of a biomolecule, preferably a nucleic acid molecule. Nucleic acids are amplified with the aid of enzymes known as polymerases. In amplification, the initial sequence is referred to as the amplicon and the product as the amplificate.

In preferred versions of the mediator probe according to the invention, the first oligonucleotide has 1 to 200, preferably 20 to 80, particularly preferred 35 to 65 nucleotides, and the mediator 1 to 60, preferably 10 to 50, particularly preferred 15 to 40 nucleotides.

According to preferred versions of the mediator probe according to the invention, the target molecule and/or the template molecule is a biomolecule selected from the group consisting of nucleic acids, DNA, RNA, peptide, protein, aptamer and/or combinations thereof.

In preferred embodiments of the present invention, the target molecule is the template molecule. In other preferred versions, the target molecule interacts with the template molecule.

In preferred versions of the mediator probe according to the invention, the 3′ terminus of the first oligonucleotide of the mediator probe serves as the starting point of an amplification reaction and can thus act as a primer. This is a decisive advantage over known state-of-the-art solutions because the mediator probe for new target molecules does not have to be completely redesigned and a probe region in the target molecule selected, but an existing primer can easily be modified in such a way that it can function as a mediator probe according to the invention.

In another preferred version of the mediator probe according to the invention, the 3′ terminus of the first oligonucleotide of the mediator probe does not serve as the starting point of an amplification reaction. In the absence of the target molecule, a corresponding mediator probe may form a hairpin structure such that it is in closed form, the mediator being bound to the first oligonucleotide of the mediator probe. In the presence of the target molecule, the mediator probe binds to the target molecule or template molecule, whereby a primer can bind the first oligonucleotide of the now opened mediator probe. By processing with a suitable enzyme system, the attached primer can be extended, whereby the mediator probe displaces the mediator. The released mediator can be detected with the help of a specific detection molecule. A preferred execution example is shown in example 18.

In a preferred design, the first oligonucleotide of a mediator probe according to the invention may comprise an aptamer region, a mediator binding region and a primer binding region. The target molecule to be detected can be a protein or peptide, for example, but is not limited to it. In the absence of the target molecule, a primer can bind to the primer binding region of the first oligonucleotide of the mediator probe and by extending the 3′ terminus of the primer using a suitable enzyme system, the mediator is released from the mediator probe. The released mediator can trigger a detectable signal using a specific detection molecule or method. In the presence of the target molecule, the aptamer region of the mediator probe binds to the target molecule, whereby the primer attached to the primer binder region cannot be prolonged and the mediator probe does accordingly not release the mediator. If the target molecule is present, a signal drop is detected in comparison to the absence of the target molecule.

Furthermore, the invention concerns a system comprising at least one mediator probe according to the invention and at least one detection molecule, characterized in that the at least one detection molecule comprises one or more oligonucleotides and comprises at least one first region which interacts with at least one mediator, and in that the at least one detection molecule comprises one or more oligonucleotides and comprises at least one first region which interacts with at least one mediator.
a) a second region comprising a fluorescence acceptor or a fluorescence donor and/or a chemical group for binding to a solid phase and/or a chemical protecting group and/or redox modifications and/or luminescence modifications, and/or
b) a third region comprising a fluorescence donor or a fluorescence acceptor and/or a chemical group for binding to a solid phase and/or a chemical protecting group and/or redox modifications and/or luminescence modifications, or
c) at least one fourth region which interacts with at least one first probe which has a fluorescence donor and/or a fluorescence acceptor, and/or
d) at least one fifth region interacting with at least one second probe comprising a fluorescence donor and/or a fluorescence acceptor or
e) consists of two oligonucleotides, both or only one of the two oligonucleotides having a fluorescence acceptor or a fluorescence donor and/or a chemical group for binding to a solid phase and/or a chemical protective group and/or redox modifications and/or luminescence modifications

The probes interacting with the at least one detection molecule can be regarded as components of the detection molecule in the sense of the invention. In this case, the detection molecule comprises more than one oligonucleotide.

The different regions of the detection molecules described here may overlap or be identical in preferred versions of invention.

Furthermore, the invention concerns a system comprising at least one mediator probe according to the invention and at least one detection molecule, characterized in that the at least one detection molecule comprises one or more oligonucleotides and comprises at least one first region which interacts with at least one mediator, and in that the at least one detection molecule comprises one or more oligonucleotides and comprises at least one first region which interacts with at least one mediator.
a) a second region comprising a fluorescence acceptor or a fluorescence donor and/or a chemical group for binding to a solid phase and/or a chemical protecting group and/or redox modifications and/or luminescence modifications, and/or
b) a third region comprising a fluorescence donor or a fluorescence acceptor and/or a chemical group for binding to a solid phase and/or a chemical protecting group and/or redox modifications and/or luminescence modifications, or
c) at least one fourth region which interacts with at least one first probe which has a fluorescence donor and/or a fluorescence acceptor, and/or
d) at least one fifth region interacting with at least one second probe comprising a fluorescence donor and/or a fluorescence acceptor.

In addition, the present invention concerns a system comprising at least one mediator probe according to the invention and at least one detection molecule, wherein the at least one detection molecule is an oligonucleotide and comprises at least one first region interacting with at least one mediator, and
a) a second region at a 5′ terminus of the at least one detection molecule which has a fluorescence acceptor or a fluorescence donor and/or a chemical group for binding to a solid phase and/or a chemical protective group and/or redox modifications and/or luminescence modifications, and
b) a third region comprising a fluorescence donor or a fluorescence acceptor and/or redox modifications and/or luminescence modifications and/or a chemical group for binding to a solid phase and/or a chemical protecting group, or
c) at least one fourth region which interacts with at least one first probe which has a fluorescence donor and/or a fluorescence acceptor, and/or
d) at least one fifth region interacting with at least one second probe comprising a fluorescence donor and/or a fluorescence acceptor.

By astutely taking advantage of the universal applicability of the mediator probe according to the invention, it is possible to use the present system for the detection of different target molecules. Using several universal mediator probes, several target molecules can be detected simultaneously in one sample.

Since the fluorescence donors and acceptors are bound to the universal detection molecule and not to target sequence-specific molecules, the fluorescence yield and the basic signal are not influenced by the structure of the target molecule. In contrast to state-of-the-art technology, an optimized detection molecule can be used in different assays without sacrificing fluorescence yield or fundamental signal.

A fluorescence acceptor or acceptor dye is a molecule that can absorb energy from a fluorescence donor. A fluorescence acceptor can also be described as a quencher in the sense of the invention. The absorption efficiency depends, among other things, on the distance between the fluorescence acceptor and the fluorescence donor. A fluorescence acceptor can be activated by absorption of a photon with λ₁ to emission with λ₂ or can be non-emissive and lead to fluorescence quenching.

A fluorescence donor is a dye molecule or fluorophore that is capable of fluorescence. A fluorescence donor, which is activated by radiation, can transfer the energy without radiation via dipole-dipole interactions to a fluorescence acceptor. This quenches the fluorescence signal of the fluorescence donor. Alternatively, the fluorescence signal of the fluorescence donor to be detected can be influenced by static and dynamic quenching.

A fluorophore (or fluorochrome, similar to a chromophore) is a fluorescent chemical compound that can re-emit light upon light triggering. Fluorophores for use as labels in constructing labeled oligonucleotides of the invention preferably comprise rhodamine and derivatives such as Texas Red, Fluorescein and derivatives such as 5-bromomethyl fluorescein, Lucifer Yellow, IAEDANS, 7-Me2N-coumarin-4-acetates, 7-OH-4-CH3-coumarin-3-acetates, 7-NH2-4CH3-coumarin-3-acetates (AMCA), monobromobimans, pyrenetrisulfonates such as Cascade Blue, and monobromotrimethylammoniobimans, FAM, TET, CAL Fluor Gold 540, HEX, JOE, VIC, CAL Fluor Orange 560, Cy3, NED, Quasar 570, Oyster 556, TMR, CAL Fluor Red 590, ROX, LC red 610, CAL Fluor Red 610, Texas red, LC red 610, CAL Fluor Red 610, LC red 640, CAL Fluor Red 635, Cy5, LC red 670, Quasar 670, Oyster 645, LC red 705, Cy5.5, BODIPY FL, Oregon Green 488, Rhodamine Green, Oregon Green 514, Cal Gold, BODIPY R6Gj, Yakima Yellow, JOE, HEX, Cal Orange, BODIPY TMR-X, Quasar-570/Cy3, TAMRA, Rhodamine Red-X, Redmond Red, BODIPY 581/591, Cy3.5, Cal Red/Texas Red, BODIPY TR-X, BODIPY 630/665-X, Pulsar-650, Quasar-670/Cy5.

“Quenching” refers to any process that reduces the fluorescence intensity of a particular substance. Quenching is the basis for Förster Resonance Energy Transfer (FRET) assays. FRET is a dynamic extinguishing mechanism because the energy transfer takes place while the donor is in an activated state. A quencher is a molecule that extinguishes fluorescence via FRET emitted by the fluorophore when activated by a light source. Quenchers for use as labels in constructing labeled oligonucleotides or probes of the invention preferably comprising DDQ-I, Dabcyl, Eclipse, TAMRA, Iowa Black FQ, BHQ-1, QSY-7, BHQ-2, DDQ-II, Iowa Black RQ, QSY-21, BHQ-3, QSY-35, BHQ-0, QSY-9, ElleQuencher, Iowa Black. The expert can select appropriate reporter quencher pairs as described in the literature [Johansson, M. K. Methods in Molecular Biology 335, 17-29 (2006); Marras, S. A. Methods in Molecular Biology 335, 3-16 (2006)].

In a preferred version of the system according to the invention, the detection molecule has a hairpin structure. Here, the hairpin structure may be formed by the 5′ terminus of the detection molecule complementarily hybridizing to an internal sequence portion and the 3′ terminus of the detection molecule comprising an unpaired sequence portion.

In addition, a detection molecule may, according to the invention, contain at least one fluorescence modification or redox modification or luminescence modification at the 5′ terminus and/or within the hairpin structure.

A hairpin structure in the sense of the present invention means a secondary structure of a linear nucleic acid molecule or oligonucleotide having sequence segments aligned by internal base pairing. These structures occur when two regions of the same molecule—usually with a palindromic nucleotide sequence—form a double helix, which is terminated at the end by an unpaired loop.

After formation of the hairpin structure, the fluorescence donor or fluorescence acceptor at the 5′ terminus (second region) and the fluorescence donor or fluorescence acceptor of the third region may interact with each other, resulting in suppression of the fluorescence signal (FRET). As an alternative to a fluorescence donor and fluorescence acceptor modification of the detection molecule in the second and third regions, other signal generating modifications may be used, such as, but not limited to, redox molecules, chemiluminescence resonance energy transfer (CRET) pairs and intercalating molecules.

After release, the mediator is diffusively present in the reaction solution and can interact with the first region of the detection molecule located at the unpaired sequence section at the 3′ terminus of the hairpin-shaped detection molecule. The detection molecule may be immobilized on a solid phase or present in solution. An extension of the mediator bound to the detection molecule can be effected by a suitable auxiliary molecule, for example the beach displacement polymerase, wherein the second region (5′ terminus) of the detection molecule, which is complementary hybridized with an internal sequence section of the detection molecule and thus forms the hairpin structure, is displaced by the polymerase or the extended 3′ terminus of the mediator, respectively. This increases the distance between fluorescence acceptor and fluorescence donor by displacing the 5′ terminus and restores the previously suppressed fluorescence signal of the fluorescence donor. Alternatively, the distance of a redox molecule at the 5′ terminus of the detection molecule changes in relation to the 3′ terminus of the detection molecule or the efficiency of CRET changes or the intercalation of molecules changes due to the formation of a duplex structure of mediator or its extension product and the detection molecule. By displacing the hybridized 5′ terminus, the formation of the secondary structure or hairpin structure is eliminated. In this case, the mediator can be extended complementarily by the described auxiliary molecule up to the 5′ terminus of the detection molecule.

According to a preferred design of the system according to the invention, the detection molecule has the structure of a molecular beacon and contains at least one mediator binding region. Molecular beacons are a special class of doubly labeled detection molecules with self-complementary strand ends that form a hairpin structure in their native state. Molecular beacons can carry labels such as a fluorescence donor and a fluorescence acceptor at the ends of the strands, whereby the labels can interact with each other in preferred versions. The hairpin structure brings the fluorescence donor and the fluorescence acceptor in close proximity to each other, thereby suppressing the fluorescence signal. Hybridization with the mediator separates the fluorescence donor and the fluorescence acceptor spatially, possibly as part of an amplification reaction in which the mediator is extended, and generates a fluorescence signal. An advantage of detection molecules in the form of molecular beacons over detection molecules that carry internal labels is lower synthesis costs for terminal labels, such as fluorescent labels.

In another preferred design, the invention concerns a system comprising at least one mediator probe according to the invention and at least one detection molecule, characterized in that the at least one detection molecule comprises two oligonucleotides, wherein
the first oligonucleotide comprises a first region interacting with at least one mediator and a second region having a fluorescence acceptor or a fluorescence donor and/or a chemical group for binding to a solid phase and/or a chemical protecting group and/or redox modifications and/or luminescence modifications, and
the second oligonucleotide comprises a third region having a fluorescence donor or a fluorescence acceptor and/or a chemical group for binding to a solid phase and/or a chemical protecting group and/or redox modifications and/or luminescence modifications,
wherein the first and second oligonucletoids have hybridizing regions with each other.

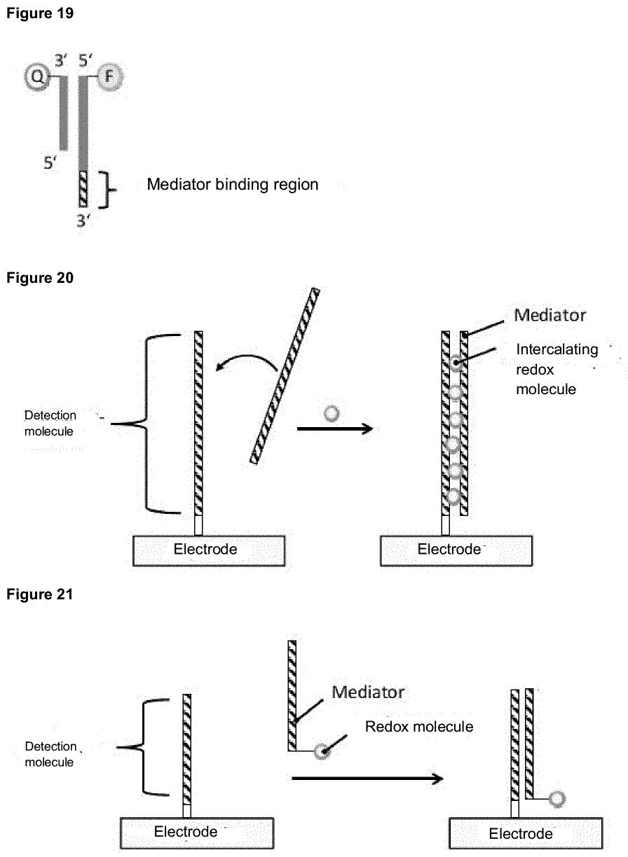

According to this design, the detection molecule may consist of two hybridized labeled oligonucleotides, whereby the two labels of the two oligonucleotides may each be at least one fluorescence acceptor or fluorescence donor attached to the ends of the oligonucleotides, whereby the fluorescence signal in the dimer is attenuated or suppressed by the spatial proximity of the two labels. One or both oligonucleotides also have a mediator binding region. By attachment of the mediator to the mediator binding region and subsequent extension, the labeled nucleotides are separated and the labeled 5′ and 3′ ends are spatially separated from each other, resulting in a detectable signal increase. An advantage of this structure consists in the very low synthesis costs for such terminal and single fluorescence labeled oligonucleotides. A preferred variant of this design of the detection molecule is shown in FIG. 19.

According to a preferred design of the invention system, the detection molecule comprises a sixth region at a 3′ terminus of the detection molecule which has a chemical group for binding to a solid phase and/or a chemical protecting group.

A protective group in the sense of this invention refers to a substituent, introduced into a molecule to temporarily protect a particular functional group and thus prevent an undesirable reaction. After the desired reaction has been carried out elsewhere on the molecule, the protective group can be split off again.

In a preferred design, the detection molecule is freely present in a solution. In another preferred design, the detection molecule is bound to a solid phase. The detection molecule is immobilized on a solid phase in a reaction vessel suitable for the respective detection method. The sample and reagents required can be added to the reaction vessel and the mixture can then be incubated under the appropriate conditions. The sample may consist of DNA, RNA and/or peptides or proteins. If the target molecule is present, the mediator is displaced or released by the mediator probe and can diffuse in the reaction mixture to the immobilized detection molecule.

In preferred versions of the invention, a signal change is detected after release of the mediator and binding to a detection molecule by electrochemical detection on a solid phase. The detection molecule is immobilized on an electrode, which simultaneously represents the solid phase. The released mediator can hybridize to the mediator binding region of the detection molecule and be extended by a polymerase, for example. After successful extension, redox molecules can intercalate into the dimer of detection molecule and extended mediator and generate an electrochemical signal that can be detected.

In addition, it is also possible that a sufficiently long mediator hybridizes with the detection molecule and is not extended. Redox molecules can intercalate into the dimer of detection molecule and mediator and generate an electrochemical signal. A variant of this design is shown in FIG. 20.

In further versions, the mediator and/or the detection molecule is labeled with one or more redox molecules. If the mediator is labeled with a redox molecule, the binding of the released mediator to the immobilized detection molecule leads to a signal change due to the spatial proximity of the redox modification and the electrode surface. A variant of this form of invention is shown in FIG. 21.

It may be advantageous to release multiple mediators per target and/or template molecule to obtain a stronger signal. The release of several mediators per target molecule can be achieved, for example, by attaching mediators to several different primers.

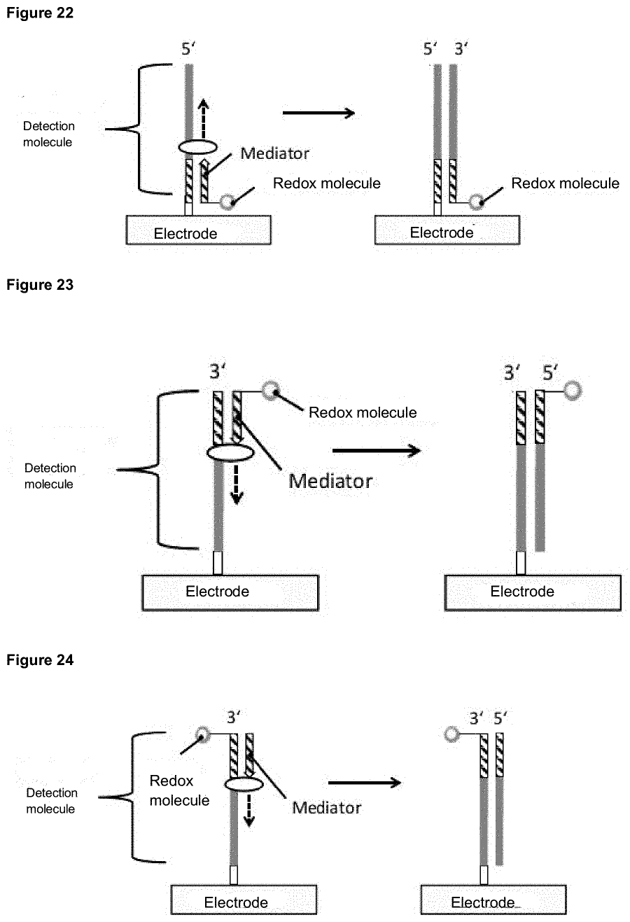

In a further version, a mediator labeled with a redox modification, for example, can be extended by a polymerase after hybridization with the detection molecule, whereby an advantageous stabilization of the double strand can be achieved (FIG. 22).

In other versions, the mediator, which is marked with a redox modification can bind to the strand end of the detection molecule removed from the electrode surface. The mediator can be extended at a longer detection molecule or the mediator already has a similar length as the detection molecule and is not extended. The electrical charge transfer between the redox molecule and the electrode takes place via the electrical conductivity of the DNA, whereby the conductivity increases through the formation of a double strand. A variant of such a design is shown in FIG. 23.

In further versions, the detection molecule may carry a redox molecule and the mediator may be label-free. The released mediator can now bind to the detection molecule and be extended as shown in FIG. 24. Alternatively, an already sufficiently long mediator can bind to the detection molecule. The formation of a double strand increases the electrical conductivity of DNA due to possible intra- and intermolecular charge transfer mechanisms. Consequently, a signal change at the electrode can be detected.

Electrochemical detection can be performed by measuring various parameters, such as impedance changes, cyclovoltammetry, square wave voltammetry or capacitance changes.

In preferred versions of the invention, the detection of a signal change after release of the mediator and binding to a detection molecule is performed via internal total reflection fluorescence microscopy (TIRF). In this design, the detection molecule is immobilized on a solid phase above a TIRF illumination device. The evanescent field formed by total reflection penetrates into the sample volume and triggers fluorescence molecules, which are located at the detection molecule and/or at the mediator and/or at probes or are intercalated in dimers, whereby a change of the fluorescence signal can be detected.

In other preferred designs, the binding of the released mediator to the detection molecule can be detected by surface plasmon resonance spectroscopy. By the release and subsequent binding of the mediator to the detection molecule immobilized on a surface, a change in the refractive index in the sample can be detected. The detection molecules can be immobilized directly on the metal surface in which the plasmons are activated or, for example, in/on a membrane located directly on the metal surface.

The binding or immobilization of the detection molecule to a solid phase is also advantageous to prove the release and binding of the mediator to a detection molecule by gravimetric measurements. For example, the detection molecule is immobilized on a carrier surface whose weight can be determined with oscillating quartz. Changes in weight due to binding of the mediator to the detection molecule can thus be detected.

Alternatively, the detection molecules can be immobilized on magnetic particles. This enables the detection of target molecules by magnetic relaxometry. In magnetic relaxometry, the magnetic particles are magnetized by a short magnetic pulse and the temporal degradation of the induced magnetic moment is detected. The hydrodynamic resistance of particles to which mediators bind and are extended via the detection molecules immobilized on the particles is greater, i.e. the hydrodynamic resistance of particles to which no mediators bind. Particles, to which mediators bind and optionally are extended, therefore degrade their induced magnetic moment more slowly than particles, to which no mediators bind. The relaxation times of the induced magnetic moments of the mentioned particles therefore differ from each other, whereby the release of mediators can be detected. By combining magnetic relaxometry with a melting curve analysis, different target molecules can be detected side by side in one sample.

According to the invention, the mediator and/or the detection molecule can be labeled with at least one fluorescent molecule, redox molecule, luminescent molecule or another signal generating molecule.

The detection molecule may be a single-stranded nucleic acid molecule or nucleic acid derivative, according to the invention. Several labeled probes can be hybridized to such a detection molecule. After release of the mediator, it binds to the detection molecule and is prolonged. This releases the labeled probes hybridized to the detection molecule, resulting in a detectable signal change emanating from the labels of the probes. For example, the release of probes labeled with fluorescence donor and fluorescence acceptor leads to an increase in fluorescence due to the spatial separation of fluorescence donor and fluorescence acceptor. The single-stranded detection molecule can be linear or circular, it can be homogeneous in solution or immobilized on a solid phase and may have several mediator binding sites. This form of invention is preferably used in isothermal amplification methods to ensure that the labeled probes bind to the detection molecule in the absence of the target molecule and do not dissociate from the detection molecule due to the high thermal energy generated, for example, by PCR. Examples of the form of the invention shown here are shown in Example 9.

If the detection molecule is circular and several mediator binding sites are inserted, a rapid detection reaction can take place in a good dynamic range by simultaneously binding several mediators at different sites. The circular structure of the detection molecule allows an additional increase in sensitivity to be achieved, since hybridization and extension of a mediator on a detection molecule releases all bound, labeled probes, regardless of the site to which the mediator binds. Probes with different fluorescence donors and fluorescence acceptors, which emit at different wavelengths can be bound to a detection molecule. By combining different fluorescent dyes, which can also be used in different concentrations (which is determined by the number of labeled probes per detection molecule), the degree of multiplexing can be increased. Certain concentration ratios can be assigned to a defined detection molecule.

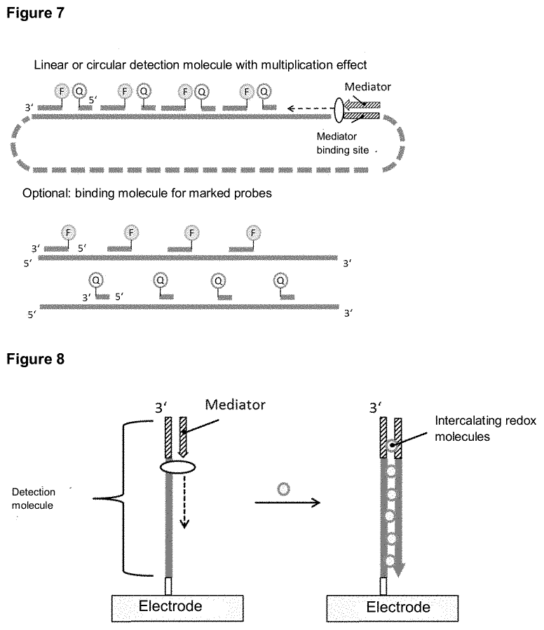

In a preferred design, the system according to the invention also includes at least one binding molecule to which at least one first and/or at least one second probe can bind after release from the detection molecule. Binding molecules can be used to prevent released probes, e.g. those labeled with fluorescence donors or fluorescence acceptors, from binding again to the detection molecule. A corresponding example of the invention is shown in FIG. 7.

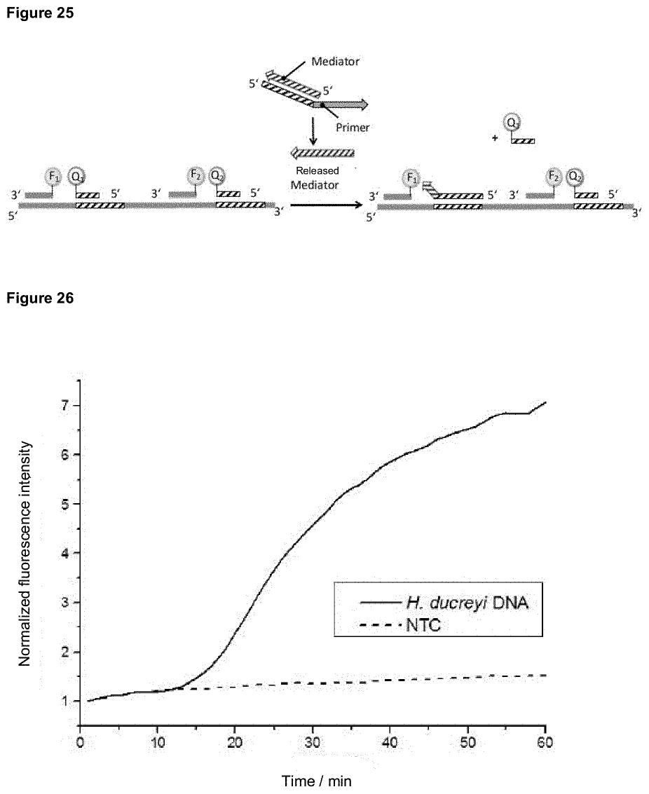

In certain forms, the detection molecule consists of several oligonucleotides, whereby an unlabeled oligonucleotide is hybridized with shorter, fluorescence-labeled oligonucleotides. The unlabeled oligonucleotide may be hybridized with several fluorescence-labeled oligonucleotides. Fluorescence acceptors and/or fluorescence donors are attached to the shorter oligonucleotides. These are arranged in such a way that the fluorophore and quencher are spatially close to each other. The released, unlabeled mediator has a higher binding energy to the unlabeled detection molecule than the fluorescence-labeled detection molecules and thus displaces, for example, the shorter oligonucleotide labeled with the quencher. The binding energies can be adjusted so that the mediator binds preferentially to the primer and not to the detection molecule under reaction conditions. A corresponding example of the invention is shown in FIG. 25.

Furthermore, the present invention concerns a method for the detection of at least one target molecule, comprising the following steps:
a) Provide at least one mediator probe according to any of claims 1 to 3 and/or a system according to claim 4 or 5,
b) Binding of the probe region of the first oligonucleotide of the at least one mediator probe to a sequence of the template molecule and/or of the target molecule,
c) Amplification of the first oligonucleotide of the at least one mediator probe and/or of the template molecule and/or of the target molecule,
d) Release of at least one mediator by at least one auxiliary molecule,
e) Optionally, binding the at least one released mediator to the at least one detection molecule, and
f) Detecting a signal change.

The amplification steps of the method according to the invention may include isothermal and/or non-isothermal amplification methods.

In isothermal or isothermal amplification, the respective reaction takes place at a constant temperature (isothermal) with a strand-shifted polymerase. Since isothermal amplification is carried out at a constant temperature, it can also be carried out without any major technical equipment effort. The strand-displacing polymerase, e.g. the Φ29 DNA polymerase from the bacteriophage Φ29, displaces an existing second strand of double-stranded DNA, while it uses the first strand to produce a new strand with the same sequence to form the second strand.

Methods for isothermal amplification of DNA include, but are not limited to, multi-displacement amplification, isothermal assembly, recombinase polymerase amplification (RPA), loop-mediated isothermal amplification (LAMP), nucleic acid sequence-based amplification (NASBA), helicase-dependent amplification (HDA), nicking enzyme amplification reaction (NEAR), rolling circle amplification (RCA) and beach displacement amplification (SDA).

In non-isothermal amplification methods, a thermostable polymerase is used because the temperature varies during the reaction. A thermal cycler can be used for this purpose. Examples of non-isothermal amplification methods are polymerase chain reaction (PCR), real-time PCR and polymerase chain displacement reaction (PCDR).

An important advantage of the method according to the invention is that the mediator release and the subsequent signal generation, for example by interaction of the mediator with a detection molecule, can be applied to different amplification methods and is not limited to specific amplification systems. By astutely taking advantage of the respective conditions in different amplification methods, the above-mentioned mediator release can easily be adapted to the respective system.

In preferred versions of the invention, the mediator is not released by splitting the mediator from the mediator probe using the nuclease activity of an enzyme, but by displacement. In this case, covalent bonds are preferably not cleaved because the mediator is not covalently bound to the first oligonucleotide of the mediator probe. Preferably, the mediator is released without the cleavage of an oligonucleotide. Preferably, the use of enzymes with nuclease activity is not required in the invention procedure.

The mediator binds to the first region of the detection molecule in a preferred version of the procedure according to the invention, whereby the binding can be an indirect or direct interaction. Through this interaction of the mediator with the first region of the detection molecule, a physical or chemically measurable change of the detection molecule can occur.

In a preferred version of the method according to the invention, at least one mediator binds to the first region of the detection molecule and is enzymatically extended by at least one auxiliary molecule, the auxiliary molecule preferably binding to the 3′ terminus of the bound mediator, whereby a physically or chemically measurable change in the detection molecule takes place. These changes of the detection molecule include, without limitation, cleavage, digestion, strand duplication, internal hybridization, phosphorylation, dephosphorylation, amidation, binding or cleavage of a chemical group, fluorescence, phosphorescence or luminescence changes.

According to another preferred version of the procedure according to the invention, at least one mediator binds to the first region of the detection molecule, which results in a physically or chemically measurable change of the detection molecule. In order to generate a measurable change, an enzymatic extension of the mediator is not necessary.

The 3′ terminus of the first oligonucleotide of the mediator probe is preferentially extended enzymatically by an auxiliary molecule after the binding of the probe region of the first oligonucleotide of the mediator probe to a sequence of the template molecule and/or the target molecule.

A preferred design of the method according to the invention is characterized in that the amplification of the first oligonucleotide of the mediator probe and/or of the template molecule and/or target molecule is carried out by an isothermal or non-isothermal amplification method.

According to the invention, PCR, PCDR or real-time PCR are preferred as non-isothermal amplification methods. LAMP or RPA are the preferred isothermal amplification methods.

In a non-isothermal amplification reaction, such as PCR or PCDR, one or more of the primers used can be modified in such a way that it is a mediator probe. Sample and reagents are placed in a suitable reaction vessel and the mixture incubated, where amplification may take place. During this process, the signal change, for example of a fluorescence signal, is detected in the reaction vessel.

In a further version of the method according to the invention, the detection molecule has at least one fluorescence or luminescence modification and, after the reaction with the at least one mediator by means of an auxiliary molecule, the fluorescence or luminescence modification is cleaved off from the detection molecule and/or the 5′ terminus of the hairpin structure of the detection molecule is removed and/or the hairpin structure is unfolded and a change in the fluorescence or luminescence signal is detected on the detection molecule.

In preferred versions of the procedure according to the invention, several mediators per mediator probe and/or several mediator probes and/or several detection molecules per target molecule are used.

This enables complex multiplex analyses that allow the simultaneous detection of several analytes in an experimental approach. Multiplex analyses enable the detection of several different target molecules and/or template molecules in a reaction mixture. In order to increase the degree of multiplexing of the method according to the invention, n different mediator probes can be used for the detection of n different target molecules. Each target molecule to be detected can be assigned at least one mediator probe whose probe region interacts specifically with the target molecule or template molecule. The mediator binding region and the mediator of the respective mediator probe are not affine or complementary to the respective target molecule or template molecule. However, the respective mediator represents a specific interaction partner for a defined detection molecule. Thus, each target molecule is indirectly assigned at least one detection molecule, which is assigned by the mediator probe. The detection of different target molecules requires different detection molecules.

Since the probe region and mediator binding region of the mediator probe can be freely combined independently of each other, a detection molecule can also be correlated with another target molecule by linking and synthesizing the matching mediator binding region and mediator with any probe region. The method according to the invention therefore allows the target molecule to be designed independently of the detection molecule. Thus, with a standardized set of detection molecules, different target molecules can be detected in one sample, whereby the reaction can be adapted cost-effectively to the respective target molecule by adapting the mediator probe and using suitable auxiliary molecules (e.g. primers or aptamers).

According to the invention, several different mediators, which are part of a single mediator probe and bind to the mediator binding region of the same first oligonucleotide of the mediator probe, can bind to several different detection molecules. By astutely combining several mediators of a mediator probe with different detection molecules, the degree of multiplexing of the procedure according to the invention can be greatly increased. The prerequisite is that the several different detection molecules generate different signals that are distinguishable and can preferably be detected in parallel or simultaneously.

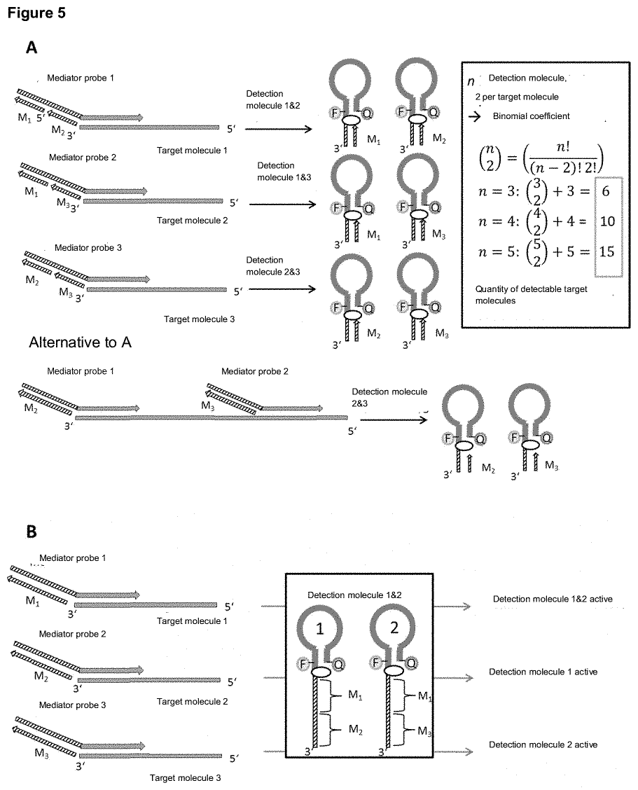

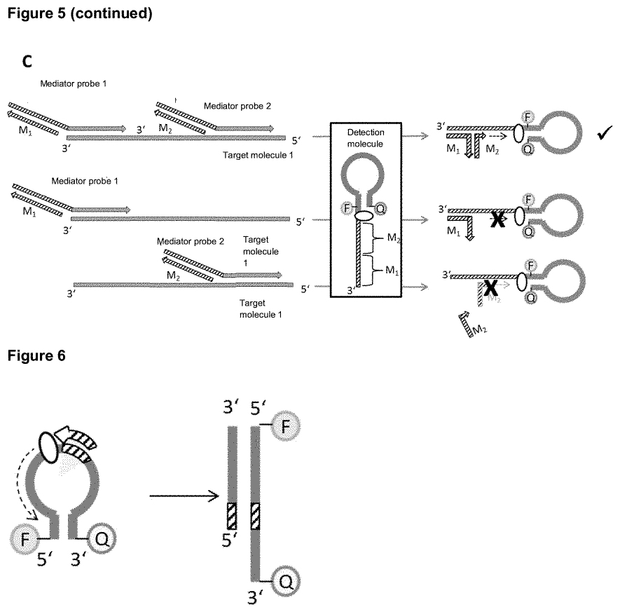

For example, by using n detection molecules and two mediators per mediator probe and target molecule “n over 2”+n different target molecules can be detected. The binomial coefficient can be used to calculate the number of detectable target molecules for a given number of different detection molecules. Since a target molecule can be identified not only by generating two different signals, for example two fluorescence signals with two different wavelengths, but also by generating a single signal, the value of the binomial coefficient must be increased by n in order to calculate the maximum number of detectable target molecules. With four different detection molecules, 10 different target molecules can be detected, while only five detection molecules can differentiate between 15 target molecules. A corresponding example is shown in FIG. 5 A.

Alternatively, several mediator probes can be used per target molecule, whereby one mediator probe can only contain one mediator.

One or more mediator probes, which bind to the same target molecule or template molecule, can be used in a preferred version of the procedure according to the invention, whereby the mediator or mediators of these mediator probes can bind to one or more detection molecules simultaneously. By astutely combining the mediator binder regions in the detection molecules, it is possible, for example, to distinguish between three target molecules using only two detection molecules. By using n detection molecules and at least two mediators per detection molecule, “2n−1” different target molecules can be detected. According to this form of invention, the detection molecules each contain at least two different mediator binding regions, whereby at least two mediators, which are linked to at least two different target sequences, each bind to only one specific detection molecule. A specific signal is generated per target molecule. According to this invention, a third or more mediators, which are linked to a third or more target sequences, can bind to at least the two detection molecules and thus trigger at least two different signals. For the probability that two released mediators simultaneously bind to the same detection molecule to be high enough, the concentration of released mediators should be in the order of the concentration of detection molecules. A corresponding example is shown in FIG. 5 B. In addition, detection molecules that can bind more than two different mediators can also be used, and several different mediator probes can bind to the same target molecule.

By using several mediator probes per target molecule or template molecule, several different mediators can be released upon detection of the target molecule. For example, several mediator probes can be used, which each bind selectively to a common target molecule or template molecule, whereby the mediator or mediators of these mediator probes have different sequences. Several different mediators of different sequences can bind to one and the same detection molecule, whereby a detection reaction can only be triggered by binding several mediators. The signal-generating reaction can be controlled by astutely taking advantage of the interaction between the released mediators and thus increasing the specificity of the detection method. Specificity refers to the proportion of events correctly classified as negative or the probability that the absence of a target molecule will also be classified as negative. For example, two mediators released by different mediator probes can interact on a detection molecule in such a way that a signal change of the detection molecule only occurs if both mediators have bound to the detection molecule. A corresponding example is shown in FIG. 5 C.

A preferred design of the method according to the invention is characterized in that the target molecule and/or the template molecule is a biomolecule selected from the group comprising DNA, RNA, peptide, protein, aptamer and/or a combination thereof.

A major advantage of the method according to the invention is that, in contrast to the state-of-the-art, the parallel detection of different molecules and molecule classes, such as proteins and nucleic acids, is possible in a single step and within a single reaction approach, thus enabling the creation of a combined DNA-RNA-protein profile of a sample.

The detection method according to the invention can be used, for example, to detect a specific RNA molecule as a target molecule, whereby the RNA is transcribed into cDNA by reverse transcription (RT) or by another suitable enzymatic system and then the cDNA is amplified, whereby the cDNA is used as a template molecule.

According to the invention, target molecule-specific aptamers can be used as template molecules for the detection of target molecules. The target molecule to be detected can be a protein or peptide, for example, but is not limited to it. An aptamer binds to the target molecule and changes its structure so that after interaction an aptamer-specific mediator probe and primer can attach to the aptamer. By processing with a suitable enzyme system, primers attached to the aptamer can be prolonged, resulting in amplification of an aptamer sequence that lies outside the protein binding region of the aptamer. A mediator probe according to the invention can interact with the aptamer or the amplified aptamer sequence. For example, by binding a mediator probe to a linear amplification product, the probe can be opened and further primers are used to displace the mediator from the mediator probe. The released mediator can be detected using a specific detection molecule or a suitable detection method. According to the invention, target molecule-specific aptamers can be used as template molecules for the detection of a target molecule, which comprise a target molecule binding region flanked by primer binding regions. In addition, at least one mediator probe according to the invention is used, the probe region of which binds specifically to one of the primer binder regions of the aptamer. In the absence of the target molecule, the target molecule binding region of the aptamer can be amplified using the mediator probe and another primer, releasing the mediator from the mediator probe and detecting a signal change. In the presence of the target molecule, the aptamer binds to the target molecule and the primer or first oligonucleotide of the mediator probe cannot be prolonged due to the binding between the aptamer and the target molecule and the mediator is not released. If the target molecule is present, a signal drop is detected in comparison to the absence of the target molecule. This method according to the invention enables an exponential detection reaction.

According to another form of the invention process, the auxiliary molecule is selected from the group consisting of polymerases, RNA polymerases, DNA polymerases, ligases, ribozymes, catalysts, proteins, nucleic acids, natural products, enzymes, enzyme systems, cell lysates, cell components, derivatives derived from cell components and/or synthetic molecules. The auxiliary molecule is preferably a molecule from a nucleic acid amplification system and/or a restriction enzyme system.

Ligases are enzymes that link DNA strands. They form an ester bond between a phosphate residue and the sugar deoxyribose. It is also known that ligases also have the ability to extend nucleic acid molecules at their 3′ end.

Ribozymes are catalytically active RNA molecules that catalyze chemical reactions like enzymes. It is known that certain ribozymes can prolong and amplify nucleic acid molecules, similar to polymerases. Ribozymes can also catalyze other reactions, such as the binding of peptide bonds and the splicing of RNA molecules.

According to a preferred version of the invention method, at least one auxiliary molecule has a DNA strand separating effect and/or a polymerizing effect, the auxiliary molecule preferably being a strand displacement polymerase.

Surprisingly, when using a polymerase with strand displacement activity, no additional enzymes, such as enzymes with nuclease activity, are required, which is a major advantage over state-of-the-art techniques.

Different auxiliary molecules can be used for different process steps within the process according to the invention. Process steps which can be carried out with the aid of auxiliary molecules comprise, without limitation, amplification of the first oligonucleotide of the mediator probe according to the invention, amplification of the target molecule and/or of the template molecule, cleavage, release or displacement of the mediator from the mediator probe, enzymatic extension of the mediator after binding to a detection molecule and modification of the detection molecule.

A measurable change in fluorescence, phosphorescence, luminescence, mass, absorption, scattering of light, electrical conductivity, enzymatic activity and/or affinity, electrochemical potential or signal, refractive index, triggering of surface plasmons or magnetic relaxation occurs in a preferred version of the method according to the invention by direct or indirect interaction between immobilized or non-immobilized detection molecule and at least one mediator.

In a preferred design of the method according to the invention, a measurable change in fluorescence, phosphorescence, luminescence, mass, absorption, scattering of light, electrical conductivity, enzymatic activity and/or affinity, electrochemical potential or signal, refractive index, triggering of surface plasmons, magnetic relaxation, magnetic property, impedance or capacitance occurs by direct or indirect interaction between immobilized or non-immobilized detection molecule and at least one mediator.

A preferred design according to the invention is characterized in that the release of the at least one mediator is detected by amplification of the at least one mediator by means of an isothermal or non-isothermal amplification method. The released mediator can, for example, trigger rolling circle amplification in the presence of corresponding amplification enzymes. Thus, the target molecule can be identified by the detection of Rolling Circle amplification products. Amplification products of Rolling Circle Amplification can, for example, be detected sequence-specifically via probes or via pH value changes, gel electrophoresis or colorimetry.

In accordance with a preferred version of the procedure according to the invention, the at least one released mediator is detected by sequencing. By sequencing the free mediators, any number of target molecules in a sample can be identified simultaneously.

Sequencing is the determination of the nucleotide sequence in a nucleic acid molecule, especially DNA. Sequencing methods within the meaning of this invention include, without limitation, the Maxam and Gilbert method, the Sanger didesoxy method, pyrosequencing, hybridization sequencing, the ion semiconductor DNA sequencing system, bridge synthesis sequencing, two-base sequencing, paired end sequencing and nanopore sequencing.

The detection can be demonstrated, for example, by next generation sequencing (NGS). An example of an NGS method is nanopore sequencing, in which potential changes on a pored membrane can be measured as molecules, such as nucleic acids, flow through the membrane and the sequence of the nucleic acid can thus be determined. Sequencing can be used to detect the simultaneous release of any number of mediators, each of which signals the presence of a specific target molecule. The degree of multiplexing increases considerably compared to conventional methods, such as fluorescence measurements. The sequencing method is not limited to nanopore sequencing.

In a preferred version of the method according to the invention, the at least one released mediator binds to the detection molecule by hybridization, is optionally extended after binding to the detection molecule by an auxiliary molecule, and then a melting curve analysis is performed. This allows an additional increase in the multiplexing degree to be achieved by using different detection molecules, which, for example, are labeled with different signaling molecules. According to a preferred version of the invention, sequence-specific or sequence-unspecific probes that are fluorogen and/or chromogen labeled or fluorescent dyes interact with the mediator and/or the detection molecule.

In a preferred version of the method according to the invention, at least one target molecule is an RNA, and the RNA is transcribed into cDNA and the cDNA serves as a template molecule. A primer with sequence overhang may be used for the rewrite reaction/reverse transcription of RNA into cDNA, and the probe region of the mediator probe may bind to a region containing both at least a portion of the cDNA and a portion of the sequence overhang.

According to another preferred version of the method according to the invention, at least one target molecule is a peptide or a protein and the template molecule is an aptamer, wherein the aptamer binds to the peptide or the protein and by binding the aptamer to the target molecule the binding site for the probe region of the mediator probe at the aptamer becomes accessible.

According to the present invention, sequence-specific or sequence-unspecific probes, fluorescent dyes or redox molecules may interact with at least one mediator and/or the detection molecule.

Furthermore, the present invention concerns the use of the system according to the invention and/or process to detect one or more similar or different biomolecules in a mixture. In this case, the detection molecule according to the invention may have a chemical protective group at the 3′ terminal region, the protective group being split off from the detection molecule by means of an auxiliary molecule after the reaction with the mediator, and a 3′ terminal OH group being generated.

Furthermore, the present invention concerns a kit comprising at least one detection molecule, and optionally at least one mediator in the sense of the invention, polymerases and dNTPs.

### SPECIAL DESCRIPTION OF THE INVENTION

In the following, the invention will be explained by means of figures and examples of execution, but without being limited to this. Show it:

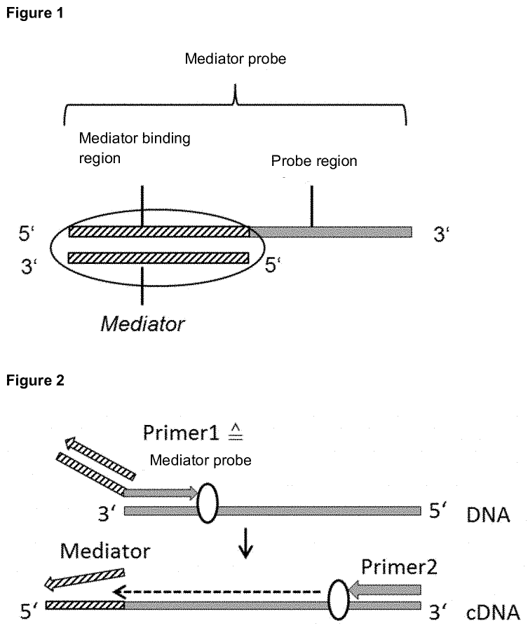

FIG. 1: Schematic representation of the structure of a mediator probe in an embodiment of the invention.

FIG. 2: Schematic sequence of a mediator displacement during an amplification process in the presence of a beach displacement polymerase.

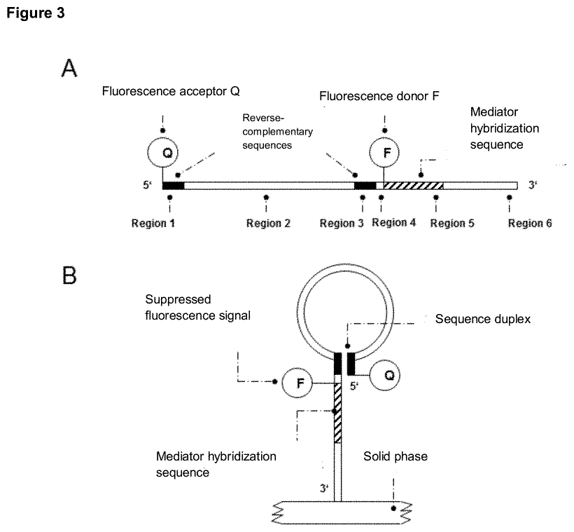

FIG. 3: Schematic representation of a possible detection molecule.

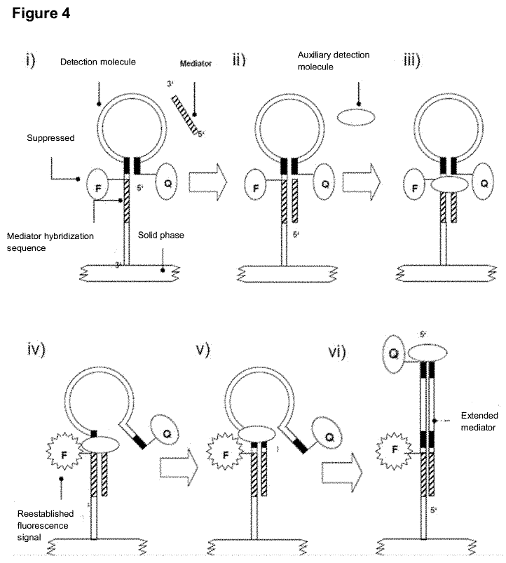

FIG. 4: Schematic representation of the enzymatic mediator extension.

FIG. 5: Arrangement possibilities when using several mediators and/or several mediator probes and/or several detection molecules per target molecule.

FIG. 6: Structure of a detection molecule with the structure of a molecular beacon.

FIG. 7: Linear or circular detection molecule with fluorescence donor and fluorescence acceptor labeled hybridized probes.

FIG. 8: Electrochemical detection on a solid phase.

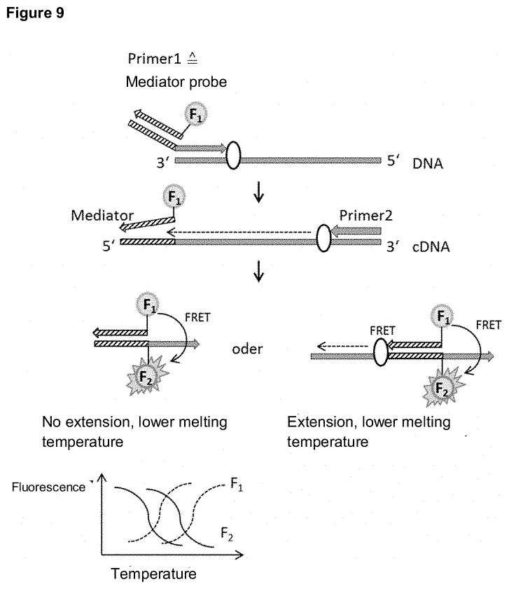

FIG. 9: Schematic sequence of a mediator displacement during an amplification process in the presence of a beach displacement polymerase.

FIG. 10: Mechanism of a mediator release and subsequent signal generation during a LAMP.

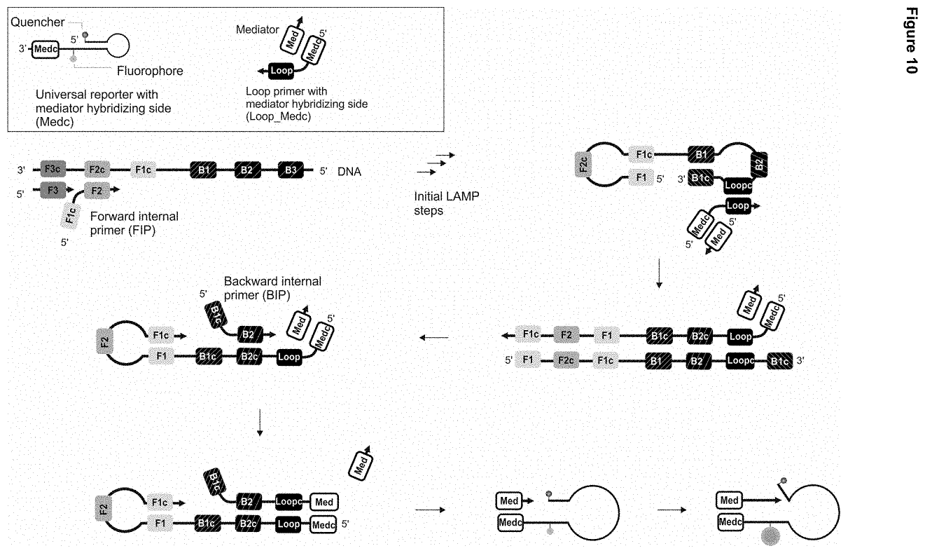

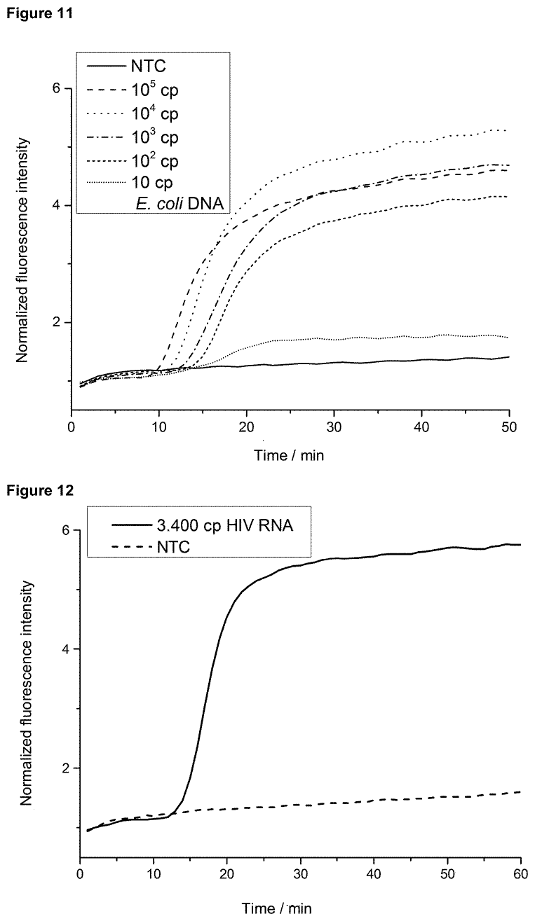

FIG. 11: Normalized fluorescence plot of a LAMP for the detection of E. coli (W3110, complete genome) using mediator probes and detection molecules according to the invention.

FIG. 12: Normalized fluorescence plot of an RT-LAMP for the detection of HIV-1 RNA using invention mediator probes and detection molecules.

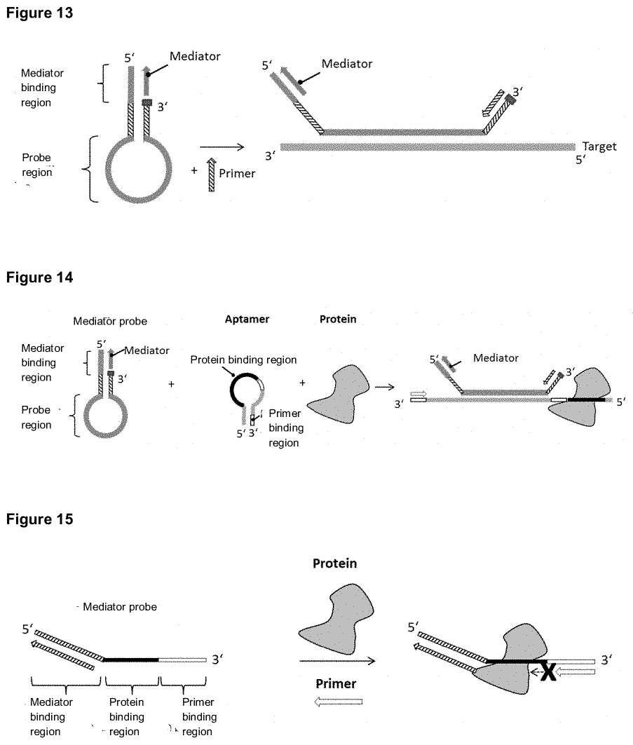

FIG. 13: Structure of a mediator probe which does not function as a primer.

FIG. 14: Detection method for the detection of target molecules by target molecule-specific aptamers.

FIG. 15: Invented detection method for the detection of target molecules by mediator probes which additionally contain aptamer region and primer binder region.

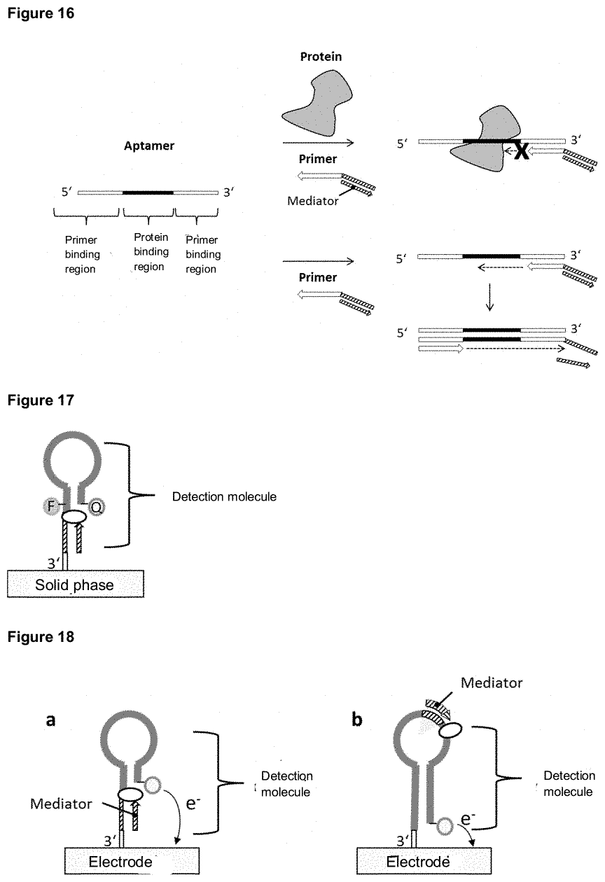

FIG. 16: Invented detection method for the detection of target molecules by mediator probes which act as primers and enable an exponential detection reaction.

FIG. 17: Immobilization of a detection molecule on a solid phase.

FIG. 18: Immobilization of a labeled detection molecule on an electrode.

FIG. 19: Detection molecule consists of two labeled, hybridized oligonucleotides.

FIGS. 20-24: Electrochemical detection on a solid phase.

FIG. 25: Detection molecule consists of several oligonucleotides.

FIG. 26: Normalized fluorescence plot of a LAMP for the detection of H. ducreyi.

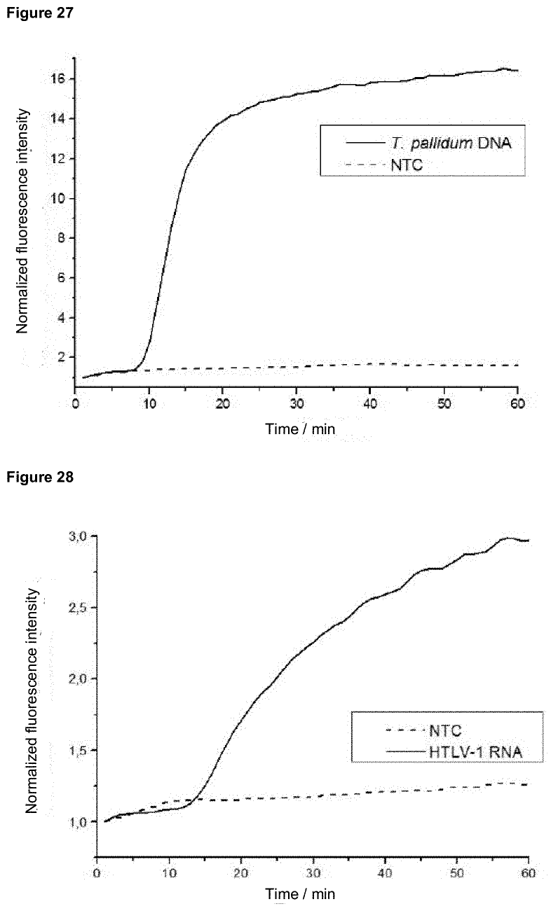

FIG. 27: Normalized fluorescence plot of a LAMP for the detection of T. pallidum.

FIG. 28: Normalized fluorescence plot of an RT-LAMP for detection of HTLV-1.

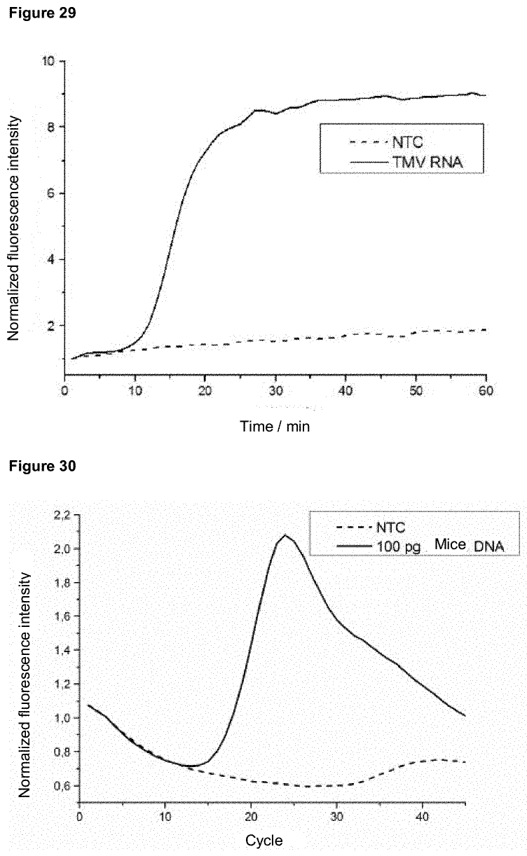

FIG. 29: Normalized fluorescence plot of an RT-LAMP for the detection of TMV.

FIG. 30: Normalized fluorescence plot of a PCDR for the detection of 100 pg G3PDH fragment.

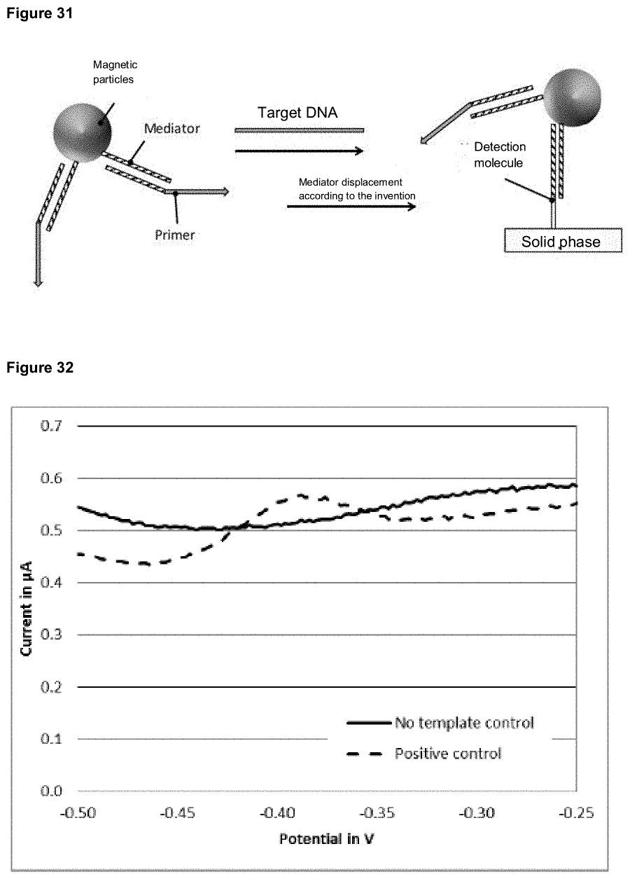

FIG. 31: Binding of the mediator to a magnetic or magnetizable nanoparticle.

FIG. 32: Proof of electrochemical detection of electroactively labeled mediators.

FIG. 1 shows a schematic representation of a possible structure of a mediator probe, which is a preferred version of the invention.

FIG. 2 shows the schematic sequence of mediator displacement during amplification in the presence of a strand displacement polymerase from a mediator probe acting as a primer in DNA amplification.

FIG. 3 (A) shows the linear representation of a possible detection molecule. (B) Representation of a 3′-immobilized detection molecule under formation of the secondary structure. The reverse-complementary sequence segments, whose interaction produces the secondary structure of the detection molecule, are shown as black regions, the mediator binding sequence as diagonally striped region.

FIG. 4 shows the schematic representation of an enzymatic mediator extension. i) A detection molecule is free in solution or immobilized on a solid phase and assumes a defined secondary structure under reaction conditions. Two suitable fluorescence modifications F and Q interact with each other, whereby the fluorescence signal of F is suppressed. ii) The mediator can interact with the detection molecule at a defined position (mediator binding region, Region 5) iii)-iv) and is thereby enzymatically extended by a strand displacement polymerase. Region 1 together with the fluorescence acceptor molecule Q is displaced by the detection molecule, thereby restoring the fluorescence intensity of the fluorescence donor F. vi) After displacement of region 1, the mediator can be further extended.

FIG. 5 (A-C) shows several possible arrangements when using several mediators and/or several mediator probes and/or several detection molecules per target molecule. (A) increasing the number of detectable target molecules using multiple mediators per mediator probe or multiple mediator probes per target molecule. The maximum number of detectable target molecules as a function of the number of detection molecules when using several mediators per target molecule can be calculated using the binomial coefficient and the number of detection molecules. (B) Increasing the number of detectable target molecules using detection molecules with multiple mediator binding regions. (C) Increasing the specificity of a detection reaction using multiple mediators per target molecule and exploiting the interaction between the mediators. The release of both mediators allows them to interact in a way with each other and simultaneously with the detection molecule, prolonging one of the mediators and triggering a detection reaction. The interaction of a single mediator does not lead to a detection reaction.

FIG. 6 shows the structure of a detection molecule corresponding to the structure of a molecular beacon. Fluorescence acceptor and fluorescence donor are attached at the 5′ and 3′ ends and the mediator binding region is located in the loop.

FIG. 7 shows a linear or circular detection molecule to which fluorescence donor and fluorescence acceptor labeled probes are hybridized. By hybridizing the mediator to the detection molecule and extension, the labeled probes are released and a signal change can be detected. In order to prevent released probes labeled with fluorescence donor or fluorescence acceptor from re binding to the detection molecule, binding molecules can be used to which the labeled probes can bind after release.

FIG. 8 shows a version of the invention in which electrochemical detection is used on a solid phase. The detection molecules are immobilized on an electrode. After hybridization and extension of the mediator at the detection molecule, redox molecules present in the solution can intercalate into the dimer of the detection molecule and the extended mediator, whereby a change in the electrochemical signal can be detected.

FIG. 9 shows the schematic sequence of mediator displacement during amplification in the presence of a strand displacement polymerase from a mediator probe acting as a primer in DNA amplification. The mediator and the detection molecule are each labeled with a fluorescent dye, which enables an increase in fluorescence intensity at one wavelength to be detected if the mediator is hybridized with the detection molecule by FRET energy transfer. When using detection molecules with different numbers of nucleotides, a melting curve analysis can be used to differentiate between different target molecules.

FIG. 10 shows the mechanism of a mediator release and subsequent signal generation during a LAMP. The mediator binding region at the detection molecule and in the mediator probe is abbreviated with Medc (corresponds to the sequence complementary to the mediator). In this version of the present invention, the mediator probe simultaneously serves as a loop primer; accordingly, the mediator probe consists of a loop primer extended by Medc (Loop_Medc) and a mediator hybridized to it (Med). After the initial LAMP steps, a dumbbell-like amplification product is formed to which the mediator probe can bind. By extending the mediator probe and reconnecting a primer to it, the mediator is displaced by the beach displacement polymerase. The released mediator can then generate a detectable signal by interacting with a detection molecule.

FIG. 11 shows a standardized fluorescence plot of a LAMP for the detection of E. coli DNA (W3110, complete genome) using mediator probes and detection molecules according to the invention. The plot shows a correlation between the amount of DNA and the fluorescence course. The fluorescence intensities were standardized to the initial value at 0 min. The number of DNA copies is expressed in copies per reaction (e.g. 10 cp corresponds to 10 copies per reaction with a total volume of 10 μl). The negative control contains 0 copies per reaction (NTC, no template control).

FIG. 12 shows the standardized fluorescence plot of an RT-LAMP for the detection of HIV-1 RNA using mediator probes and detection molecules according to the invention. The fluorescence intensities were standardized to the initial value at 0 min. The number of RNA copies is given in copies per reaction (3,400 cp corresponds to 3,400 copies per reaction with a total volume of 10 μl). The negative control contains 0 copies per reaction (NTC, no template control).

FIG. 13 shows a design of a mediator probe which does not serve as a starting point for amplification.

FIG. 14 shows a detection according to the invention method for the detection of target molecules by target molecule-specific aptamers.

FIG. 15 shows a detection according to the invention method for the detection of target molecules by mediator probes which additionally contain aptamer region and primer binder region. In the absence of the target molecule, the mediator probe is amplified, while in the presence of the target molecule, the extension of the primer is blocked by the bound target molecule. Consequently, no detectable signal is triggered in the presence of the target molecule and a detectable signal is generated in the absence of the target molecule.

FIG. 16 shows a detection method according to invention for the detection of target molecules by mediator probes, which act as primers and enable an exponential detection reaction. In the absence of the target molecule, the linear aptamer is amplified and the mediator is released during amplification, while in the presence of the target molecule, the extension of the primer is blocked by the bound target molecule. Consequently, no detectable signal is triggered in the presence of the target molecule and a detectable signal is generated in the absence of the target molecule.

FIG. 17 shows a design of the detection method according to the invention, in which the detection molecules are immobilized in a suitable reaction vessel on a solid phase.

FIG. 18 shows a version of the detection according to the invention method in which the detection molecules are immobilized on an electrode. Through hybridization and extension of the mediator at the detection molecule, the redox molecule bound to the detection molecule is spatially separated from the electrode surface, generating a change in the signal. Schemes a and b schematize possible binding sites of the mediator in two different regions of the detection molecule.

FIG. 19 shows a detection molecule consisting of two labeled, hybridized oligonucleotides. Fluorescence acceptor and fluorescence donor are attached at the 5′ and 3′ ends, respectively; in addition, one of the two oligonucleotides has a mediator binding region.

FIG. 20 shows a possibility of electrochemical detection on a solid phase. The detection molecules are immobilized on an electrode. After hybridization of the mediator at the detection molecule, redox molecules present in the solution can intercalate into the dimer of detection molecule and mediator, whereby a change in the electrochemical signal can be detected.

FIG. 21 shows electrochemical detection on a solid phase using a marked mediator. The detection molecules are immobilized on an electrode. By hybridizing the labeled mediator to the detection molecule, a change in the electrochemical signal can be detected.

FIG. 22 shows the electrochemical detection on a solid phase. The detection molecules are immobilized on an electrode. By hybridization and extension of the labeled mediator on the detection molecule, a change in the electrochemical signal can be detected.

FIG. 23 shows a possibility of electrochemical detection on a solid phase. The detection molecules are immobilized on an electrode. By hybridization and extension of the labeled mediator on the detection molecule, a change in the electrochemical signal can be detected.

The electrical charge transport between the redox molecule and the electrode is caused by the formation of a double strand.

FIG. 24 shows electrochemical detection on a solid phase. The detection molecules are immobilized on an electrode. By hybridization and extension of the mediator on the labeled detection molecule, a change in the electrochemical signal can be detected. The electrical charge transport between the redox molecule and the electrode is caused by the formation of a double strand.

FIG. 25: The detection molecule consists of several oligonucleotides in which an unlabeled oligonucleotide is hybridized with shorter fluorescence-labeled oligonucleotides. Fluorescence acceptors and/or fluorescence donors are attached to the shorter oligonucleotides. These are arranged in such a way that the fluorophore and quencher are spatially close to each other. The released mediator has a higher binding energy to the unlabeled detection molecule and thus displaces, for example, the shorter oligonucleotide labeled with the quencher.

FIG. 26 shows a standardized fluorescence plot of a LAMP for the detection of Haemophilus ducreyi (H. ducreyi) using mediator probes and detection molecules according to the invention. The fluorescence intensities were standardized to the initial value at 0 min. The negative control (NTC, no template control) does not contain H. ducreyi DNA, the positive control was mixed with purified H. ducreyi DNA.

FIG. 27 shows a standardized fluorescence plot of a LAMP for the detection of Treponema pallidum (T. pallidum) using mediator probe according to the inventions and detection molecules. The fluorescence intensities were standardized to the initial value at 0 min. The negative control (NTC, no template control) does not contain T. pallidum DNA, the positive control was mixed with purified T. pallidum DNA.

FIG. 28 shows a standardized fluorescence plot of an RT-LAMP for the detection of HTLV-1 using mediator probe according to the inventions and detection molecules. The fluorescence intensities were standardized to the initial value at 0 min. The negative control (NTC, no template control) does not contain HTLV-1 RNA, the positive control was mixed with purified HTLV-1 RNA.

FIG. 29 shows a standardized fluorescence plot of an RT-LAMP for the detection of TMV using mediator probe according to the inventions and detection molecules. The fluorescence intensities were standardized to the initial value at 0 min. The negative control (NTC, no template control) does not contain TMV RNA, the positive control was mixed with purified TMV RNA.

FIG. 30 shows a standardized fluorescence plot of a PCDR for the detection of 100 pg mice G3PDH DNA using mediator probe according to the inventions and detection molecules. The fluorescence intensities were normalized to the initial value at 0 cycles.

FIG. 31 shows a mediator bonded to a magnetic or magnetizable nanoparticle. After release in the presence of the target molecule, the mediator can bind to the detection molecule, detecting a change in the magnetic property on the surface of the solid phase.

FIG. 32 shows a functional demonstration of the electrochemical detection of electroactively labeled mediators. In the positive control (60,000 copies of E. coli DNA), the mediators are displaced during the LAMP reaction and can then hybridize to the detection molecule. Accordingly, the marked (here methylene blue) mediator accumulates on the electrode surface, which leads to the formation of a characteristic peak at −0.39 V in electrochemical analysis (here square wave voltammetry). In contrast, the absence of a peak at the NTC indicates that no significant release of mediators has occurred.

### DESIGN EXAMPLES

In the following explanations, several mediators can bind to a special primer or first oligonucleotide of the mediator probe according to the invention and/or several different primers and/or mediator probes can be provided with mediators in order to increase the mediator concentration in the sample.

### Example 1: Mediator Probe

Invention design examples include a mediator probe for detecting at least one target molecule, wherein the mediator probe comprises at least two oligonucleotides. A first oligonucleotide has a mediator binding region and a probe region. The mediator binding region is located at the 5′ terminus and the probe region at the 3′ terminus of the oligonucleotide. A second or several further oligonucleotides, the mediator or mediators, are chemically, biologically and/or physically bound to the mediator binding region of the first oligonucleotide. A mediator can be composed of DNA, RNA, PNA or modified RNA, such as LNA. The probe region of the first oligonucleotide has an affinity to the target and/or template molecule and the mediator binding region has an affinity to the mediator or mediators (FIG. 1). The mediator or mediators have an affinity for at least one detection molecule.

### Example 2: Procedure of Mediator Displacement

After binding of the probe region to a target molecule and/or template molecule, the mediator is displaced by the mediator binding region, for example using a beach displacement polymerase. This process can take place during an amplification process of the target molecule and/or template molecule. In the examples of the invention, the probe region of the mediator probe can act as a primer in DNA amplification. After binding the probe region to a target molecule and/or template molecule, the mediator probe is extended. A second primer can then be attached to the extended mediator probe and extended. During the amplification process, the mediator or mediators are released from the mediator binding region and trigger a detectable signal through interaction with one or more detection molecules (FIG. 2).

### Example 3: Detection Molecule with 6 Regions

The detection of the released, unmarked mediator takes place with the help of a detection reaction. The reaction mechanism described below can be performed in parallel with the amplification of the target molecule and/or template molecule described above.

In a preferred version of the invention, a detection molecule may consist of an oligonucleotide divided into six regions (FIG. 3). Region 1 comprises the 5′ terminus of the detection molecule consisting of a sequence portion and a fluorescence acceptor Q. Region 3 is a reverse-complementary sequence of Region 1 and is separated therefrom by Region 2. Region 4 separates Region 3 and Region 5, which can specifically interact with a mediator molecule. Region 6 comprises the 3′-terminal sequence region, which may have a chemical modification and thus allows directional immobilization of the oligonucleotide. A fluorescence donor F is associated in a suitable way with a region of Region 2 to Region 6, for example Region 4. Region 1 and Region 3 of the detection molecule form a defined secondary structure (hairpin structure) under reaction conditions, in which the 5′ terminus hybridizes to an internal sequence section (FIG. 3 B). After formation of this structure, fluorescence donor F and fluorescence acceptor Q interact with each other and the fluorescence signal of F is suppressed (FRET). As an alternative to a fluorescence donor and fluorescence acceptor modification of the detection molecule in Region 1 and Region 4, other signal-generating modifications may be used, such as redox molecules, chemiluminescence resonance energy transfer (CRET) pairs and intercalating molecules.

### Example 4: Mediator Extension Leads to Signal Change of the Detection Molecule

After release, the mediator is diffusively present in the reaction solution and can interact with the mediator binding sequence (Region 5) of the detection molecule (FIG. 4 i)+ii)). The detection molecule may be immobilized on a solid phase or present in solution. The mediator is elongated by a suitable auxiliary molecule, e.g. the beach displacement polymerase, whereby Region 1 of the detection molecule is displaced by the polymerase. The distance between fluorescence acceptor Q and fluorescence donor F is increased by displacement of the 5′ terminus and the previously suppressed fluorescence signal of the fluorescence donor F is restored (FIG. 4 iii)+iv)). Alternatively, the distance of a redox molecule at the 5′ terminus of the detection molecule changes in relation to the 3′ terminus of the detection molecule or the efficiency of CRET changes or the intercalation of molecules changes due to the formation of the duplex of mediator or its extension product and the detection molecule. If the described displacement prevents the interaction of Region 1 and Region 3, the formation of the secondary structure is cancelled. In this case, the mediator can be extended complementarily by the described auxiliary molecule under certain conditions up to the newly formed 5′ terminus of the detection molecule (FIG. 4 v)+vi)). This full extension provides the extended mediator with a sequence segment complementary to Region 1, 2 and Region 3 of the detection molecule.

The detection reaction must be designed in such a way that, in contrast to the mediator, the initial mediator probe does not trigger a signal-generating reaction and thus no false-positive results are produced. Initially, the mediator is bound to the first oligonucleotide of the mediator probe, for example by hydrogen bonds. In order to prevent a signal generating reaction by binding the mediator to the detection molecule, the balance between binding the mediator to the first oligonucleotide of the mediator probe and binding the mediator to the detection molecule can be adjusted accordingly.

The interaction event of the mediator with the detection molecule produces a local, detectable signal. If a sufficient number of detection molecules are activated by the mediator extension with resulting displacement of the 5′ terminus, the signal is amplified and can be detected using suitable detection devices. This allows detection in the presence of the reaction mixture and does not require any processing steps.

### Example 5: Multiplex Analyses

Multiplex analyses require the detection of several different analytes in a reaction mixture. In order to increase the degree of multiplexing of the reaction according to the invention, the use of n different mediator probes for n different target molecules is planned. Each target molecule to be detected can be assigned a mediator probe whose probe region interacts specifically with the target molecule or template molecule. The mediator binding region and the mediator of the respective mediator probe are not affine or complementary to the target or template molecule. However, the mediator represents a specific interaction partner for a defined detection molecule. Thus, each target molecule is indirectly assigned a detection molecule, which is assigned by the mediator probe. The detection of different target molecules requires different detection molecules.

Since the probe region and mediator binding region of the mediator probe can be freely combined independently of each other, a detection molecule can also be correlated with another target molecule by linking and synthesizing the matching mediator binding region and mediator with any probe region. The method according to the invention therefore allows the target molecule to be designed independently of the detection molecule. Thus, with a standardized set of detection molecules, different target molecules can be detected in one sample, whereby the reaction can be adapted cost-effectively to the respective target molecule by adapting the mediator probe and using suitable auxiliary molecules (e.g. primers or aptamers).

Examples of invention execution may include multiple mediators and/or multiple mediator probes and/or multiple detection molecules per target molecule. The following constellations are possible.
A. Several mediators, which bind to the same mediator probe can bind to several detection molecules. By astutely combining several mediators of a mediator probe with different detection molecules, the multiplexing degree of an assay can be greatly increased. The prerequisite is that the detection molecules generate fluorescence signals with different wavelengths. By using n detection molecules and two mediators per mediator probe and target molecule, “n over 2”+n different target molecules can be detected. The binomial coefficient can be used to calculate the number of detectable target molecules for a given number of different detection molecules. Since a target molecule can be identified not only by generating two fluorescence signals with two different wavelengths, but also by generating a single fluorescence signal, the value of the binomial coefficient must be increased by n in order to calculate the maximum number of detectable target molecules. With four different detection molecules, 10 different target molecules can be detected, while only five detection molecules allow the differentiation of 15 target molecules (FIG. 5 A). Similarly, several mediator probes can be used per target molecule, whereby one mediator probe can only contain one mediator.
B. One or more mediator probes binding to the same target or template molecule may be used and the mediator or mediators of such mediator probes may bind to one or more detection molecules simultaneously. By astutely combining the mediator binder regions in the detection molecules, it is possible, for example, to distinguish between three target molecules using only two detection molecules. By using n detection molecules and at least two mediators per detection molecule, “2n−1” different target molecules can be detected. Several different mediator probes can bind to the same target molecule. In the above example, where three target molecules can be distinguished using only two detection molecules, the detection molecules may each contain two different mediator binding regions. Two mediators, which are linked to two different target sequences, each bind to only one specific detection molecule. A specific signal is generated per target molecule. The third mediator, which is linked to the third target sequence, binds to both detection molecules and thus triggers two different signals. To ensure that the probability that two released mediators bind to the same detection molecule simultaneously is high enough, the concentration of released mediators should be in the order of the concentration of detection molecules (FIG. 5 B). In addition, detection molecules that can bind more than two different mediators can also be used.
C. Several mediator probes, each selectively binding to a common target molecule or template molecule, can be used, whereby the mediator or mediators of these mediator probes have different sequences. Several different mediators can bind to one and the same detection molecule, whereby a detection reaction can only be triggered by binding several mediators. This method can be used to increase the specificity of the detection reaction. A possible reaction sequence is shown in FIG. 5 C. To ensure that the probability that two released mediators bind to the same detection molecule simultaneously is high enough, the concentration of released mediators should be in the order of the concentration of detection molecules.

### Example 6: Melt Curve Analysis

In certain versions of the invention, a melting curve analysis of the detection molecules hybridized with the extended mediators can be performed after the amplification reaction. This allows an additional increase in the multiplexing degree to be achieved by using different detection molecules, which, for example, are labeled with different signaling molecules.

### Example 7: Detection Molecule in the Form of a Molecular Beacon

In a possible form of the invention, the detection molecule has the structure of a molecular beacon in which the mediator binding region is located in the loop (FIG. 6). By attachment of the mediator to the described detection molecule and subsequent extension, the molecular beacon is opened and the labeled 5′ and 3′ ends separated, resulting in a detectable signal increase. An advantage of this structure over the structure considered so far (FIG. 3) is lower synthesis costs for terminal fluorescent markings.

### Example 8: Detection Molecule Consisting of Two Labeled Oligonucleotides

In another version of the invention, the detection molecule consists of several fluorescence-labeled oligonucleotides. Two oligonucleotides labeled with quencher and fluorophore can be hybridized with each other and separated when interacting with a mediator, thus a signal change can be detected. The described detection molecule can be structured as shown in FIG. 19.

### Example 9: Detection Molecule from Single-Stranded DNA with Hybridized Probes

In this design, the detection molecule may consist of single-stranded DNA to which several probes labeled with fluorescence donor and fluorescence acceptor are hybridized. After release of the mediator, the mediator binds to the detection molecule and is prolonged, whereby the labeled probes are released and fluorescence donor and fluorescence acceptor are spatially separated from each other, leading to an increase in fluorescence. The multiple hybridization of the detection molecule with several labeled probes leads to a multiplication effect of the signal generation. The detection molecule can be linear or circular, it can be homogeneous in solution or immobilized on a solid phase and may have several mediator binding sites. If the detection molecule is circular and several mediator binding sites are inserted, a rapid detection reaction can take place in a good dynamic range by simultaneously binding several mediators at different sites. The circular structure of the detection molecule allows an additional increase in sensitivity to be achieved, since hybridization and extension of a mediator on a detection molecule releases all bound, labeled probes, regardless of the site to which the mediator binds. Probes with different fluorescence donors and fluorescence acceptors, which emit at different wavelengths, can be bound to a detection molecule. By combining different fluorescent dyes, which can also be used in different concentrations (which is determined by the number of labeled probes per detection molecule), the degree of multiplexing can be increased. Certain concentration ratios can be assigned to a defined detection molecule. Binding molecules to which the labeled probes can bind after release (FIG. 7) can be used to prevent released probes labeled with fluorescence donor or fluorescence acceptor from re binding to the detection molecule in the long term. The described design is preferably used with isothermal amplification methods, thus ensuring that the labeled probes bind to the detection molecule in the absence of the target molecule and do not dissociate from the detection molecule due to high thermal energy generated, for example, by PCR.

In a preferred design, the probes labeled with fluorescence donor and fluorescence acceptor are not separated from the detection molecule by extending the mediator, but are displaced by adjusting the equilibrium in the presence of released mediators. The released mediator has a higher binding energy to the unlabeled detection molecule than the labeled probe and thus displaces, for example, the shorter probe labeled with the fluorescence acceptor (FIG. 25).

### Example 10: Detection Via Internal Total Reflection Fluorescence Microscopy (TIRF) or Surface Plasmon Resonance Spectroscopy

Similar to electrochemical detection, detection by internal total reflection fluorescence microscopy (TIRF) can be performed in certain designs. In this method, the detection molecule is immobilized on a glass or polymer test carrier above the TIRF illuminator. The evanescent field formed by total reflection penetrates into the sample volume and activates fluorescence molecules, which are located at the detection molecule and/or at the mediator and/or at probes or are intercalated in dimers, whereby a change of the fluorescence signal can be detected. In further versions, the binding of the mediator to the detection molecule is detected by surface plasmon resonance spectroscopy. By the release and subsequent binding of the mediator to the detection molecule immobilized on a surface, a change in the refractive index in the sample can be detected. The detection molecules can be immobilized directly on the metal surface in which the plasmons are activated or, for example, in/on a membrane located directly on the metal surface.

### Example 11: Verification by Gravimetric Measurements

In preferred versions of the invention, the release and binding of the mediator to a detection molecule can be demonstrated by gravimetric measurements. For example, the detection molecule is immobilized on a carrier surface whose weight can be determined with oscillating quartz. Changes in weight due to binding of the mediator to the detection molecule can thus be detected.

### Example 12: Proof of Rolling Circle Amplification

In preferred versions of the invention, the released mediator in the presence of appropriate amplification enzymes can trigger rolling circle amplification and thus the target molecule can be identified by the detection of the rolling circle amplification products. Amplification products of Rolling Circle Amplification can, for example, be detected sequence-specifically via probes or via pH value changes, gel electrophoresis or colorimetry.

### Example 13: Detection Via Sequencing

In preferred versions of the invention, the released mediator can be analyzed by sequencing and thus identified. An example of next-generation sequencing is nanopore sequencing, in which potential changes on a membrane with pores are measured as molecules, such as nucleic acids, flow through it, and the sequence of the nucleic acid can thus be determined. Sequencing can be used to detect the simultaneous release of any number of mediators, each of which signals the presence of a specific target molecule. The degree of multiplexing increases considerably compared to conventional methods, such as fluorescence measurements. The sequencing method is not limited to nanopore sequencing because any sequencing method can be selected for the detection of released mediators.

### Example 14: Using a Selected Mediator

In preferred versions of the invention, the mediator bound to the mediator probe can be marked with a fluorescence donor/fluorescence acceptor, which emits at a certain wavelength λ₁. After displacement of the mediator probe, the mediator can bind to a detection molecule labeled with a fluorescence acceptor/fluorescence donor, which is emitted at a second wavelength λ2, which is different to λ₁. The energy transfer from the fluorescence donor to the fluorescence acceptor via the FRET mechanism leads to a detectable increase in the radiation intensity of the fluorescence acceptor, which allows the emission of λ₂ to be detected. In further versions, chemiluminescent or bioluminescent donor molecules can be used. Fluorescence acceptors, which are non-emissive, can also be used in designs. By using detection molecules with different numbers of nucleotides, different target molecules can be distinguished simultaneously in one sample by means of a melting curve analysis. By marking the mediator, the universal character of the described detection method is not lost, since the mediator is a universal molecule independent of the target sequence. In further designs, several probes or primers per target molecule can be used to which mediators labeled with fluorescent dyes are bound, whereby the sequences of the mediators and the emission wavelengths of the fluorescent dyes may differ (FIG. 9).

### Example 15: Use of an Isothermal Amplification Method

In this example, the detection method according to invention is used for the detection of DNA in an isothermal amplification method, for example the LAMP. The mechanism of a mediator release during a LAMP is detailed in FIG. 10. The initial amplification steps of a LAMP lead to a dumbbell-like structure of an intermediate amplification product. The mediator probe, which acts as primer in this example, can bind to this intermediate amplification product and be extended in a next step. By displacing the intermediate amplification product, another primer can bind to the extended mediator probe and be extended. During this process, the mediator is displaced by the beach displacement polymerase. The released mediator can now bind to a detection molecule with a hairpin structure and can also be extended. During extension of the mediator, the 5′ end of the closed hairpin structure of the detection molecule is displaced from the complementary region, generating a fluorescence signal.

Place the sample and reagents in a suitable reaction vessel and incubate the mixture (between 10 min and 60 min at about 62° C.). During this process the fluorescence is detected in the reaction vessel. In the following, the execution described using the example of a LAMP is described in detail:

For real-time LAMP detection of E. coli DNA (W3110, complete genome), the primers listed in Table 1 were used. The LAMP primers were taken from (Tanner et al. 2012) and partially modified. The mediator probe was combined with the LoopF primer by adding a mediator binding region to the 5′ end of the primer, which can hybridize with a mediator. Mediator, LoopF with mediator binding region and the detection molecule were created manually using VisualOMP (DNA Software, USA). The synthetic oligonucleotides from Table 1 were synthesized by Biomers (biomers.net, Ulm, Germany).

TABLE 1
Sequences of primer, mediator and detection molecule for a real-
time LAMP for the detection of E. coli DNA.

| | |
|---|---|
| FIP | CTGCCCCGACGACGATAGGGCTTAATTAATCGTGGTGGTGGTGGTGTCT [symbol] |
| c | CCAGTGCGACCTGCTGGGGGTGTGTATTGTTCGCCGCCAGT |
| | AC |
| F3 | GATCACCGATTTCACCAACC |
| B3 | CTTTTGAGATCAGCAACGTCAG |
| LoopF | &LT;FONT COLOR=″#FFFF00″&GT;-==-SYNC:βçÈÂÈÂ |
| LoopB | TGAGTTAACCCACCTGACG |
| mediator | TCCGCAGCAAGTGGGGGGGCTCTACGACC |
| LoopF with | GGTCGTAGAGAGCCCACTTGCTGGCGGATGCGCCATGTCCCGCT |
| mediator | |
| binding | |
| sequence | |
| Detection | BHQ-2-GACCGGCCCCAAGACGCGCCGGGT(dC-Cy5)TGTTGGTCGT- |
| molecule | AGAGCCCAGAACGA |

[symbol]

indicates data missing or illegible when filed

The LAMP reaction was performed with Bst 2.0 WarmStart DNA Polymerase in 1× Isothermal Amplification Buffer (New England Biolabs, Frankfurt, Germany). 1× Isothermal Amplification Buffer contains 20 mM Tris-HCl, 10 mM (NH4)_{2SO4}, 50 mM KCl, 2 mM MgSO4 and 0.1% Tween® 20 (pH 8.8 at 25° C.). In addition, MgSO4 (New England Biolabs, Frankfurt, Germany), final concentration 8.0 mM, and dNTP Mix (Qiagen, Hilden, Germany), final concentration 1.4 mM, were added to the buffer. The LAMP reaction consisted of 1.6 μM FIP and BIP, 0.2 μM F3 and B3, 0.8 μM LoopB, 0.6 μM LoopF, 0.2 μM LoopF with mediator binding region, 0.1 μM Mediator, 0.05 μM detection molecule, 320 U/ml Bst 2.0 WarmStart DNA Polymerase, 1× Isothermal Amplification Buffer and 1 g/l BSA. The reaction was carried out in a rotor gene 6000 (Corbett, Mortlake, Australia, now Qiagen, Hilden, Germany) at 62° C. in triplicates. The fluorescence data were normalized to the initial value at 0 min (FIG. 11).

The detection method was also used in a LAMP of Haemophilus ducreyi (H. ducreyi) and Treponema pallidum (T. pallidum). The performance and reaction conditions were identical to the LAMP of E. coli described above, but with sequence-specific primers for H. ducreyi and T. pallidum. The fluorescence data were normalized to the initial value at 0 min (FIGS. 26 and 27).

### Example 16: Procedure According to Invention Using an RT-LAMP

In further examples, the detection according to the invention method can be used to detect RNA, whereby the RNA is transcribed into cDNA using reverse transcription (RT) or another suitable enzymatic system and the cDNA is then amplified. In the following, the execution example using an RT-LAMP is described in detail:

The primers listed in Table 2 were used for an RT-LAMP for the detection of HIV-1 RNA. The RT-LAMP primers were taken from (Curtis et al. 2008) and partially modified. The mediator probe was combined with the LoopF primer by adding a mediator binding region to the 5′ end of the primer, which can hybridize with a mediator. Mediator, LoopF with mediator binder region and the detection molecule were created manually using VisualOMP. The synthetic oligonucleotides Mediator, LoopF with mediator binding sequence and detection molecule were synthesized by Biomers (biomers.net, Ulm, Germany) and the primers FIP, BIP, F3, B3, LoopF and LoopB were synthesized by Ella Biotech (Martinsried, Germany). The template RNA (HIV, VR-3245SD) was synthesized by ATCC, LGC Standards GmbH (Wesel, Germany).

TABLE 2
Primer, mediator and detection molecule sequences for a real-time
RT-LAMP for the detection of HIV-1 RNA.

| | |
|---|---|
| FIP | CAGCTTCCTCCTCATTGATGGTTTTTTTTTTTTTTAACACCATGCTAAACA |
| | CACAGT |
| BIP | TGTTGCACCAGGCCAGATAATTTTT GTACTGGTAGTTCCTGCTATTTG |
| F3 | ATTATCAGAAGGAGGAGACCACC |
| B3 | CATCCTATTTGTTCCTGAAGG |
| LoopF | TTTAACATTTGCATGGCTGCTTGAT |
| LoopB | GAGATCCAAGGGGAAGTGA |
| Mediator | CCATGCCTCAGGAGGAGCTCAGTTCGGTCAGTG |
| LoopF with | CACTGACCGAACTGAGCTCCTGAGGGCATGGTTTAACATTTTTGCATGG |
| mediator | CTGCTTGAT |
| binding | |
| sequence | |
| Detection | BMN-Q-535-CACCGGGCCAAGACGCGCCGGGG(dT-Atto- |
| molecule | 647N)GTGTTCACT-GACCGAACTGGGAGCA |

The RT-LAMP reaction was performed with Bst 2.0 WarmStart DNA Polymerase (New England Biolabs, Frankfurt, Germany) and Transcriptor Reverse Transcriptase (Roche Diagnostics, Mannheim, Germany) in 1× Isothermal Amplification Buffer (New England Biolabs, Frankfurt, Germany). 1× Isothermal Amplification Buffer contains 20 mM Tris-HCl, 10 mM (NH4)_{2SO4}, 50 mM KCl, 2 mM MgSO4 and 0.1% Tween® 20 (pH 8.8 at 25° C.). In addition, MgSO4 (New England Biolabs, Frankfurt, Germany), final concentration 8.0 mM, and dNTP Mix (Qiagen, Hilden, Germany), final concentration 1.4 mM, were added to the buffer. The RT-LAMP reaction consisted of 1.6 μM FIP and BIP, 0.2 μM F3 and B3, 0.8 μM LoopB, 0.6 μM LoopF, 0.2 μM LoopF with mediator binding region, 0.1 μM Mediator, 0.05 μM detection molecule, 320 U/ml Bst 2.0 WarmStart DNA Polymerase, 400 U/ml Transcriptor Reverse Transcriptase and 1× Amplification Buffer. The reaction was carried out in a rotor gene 6000 (Corbett, Mortlake, Australia, now Qiagen, Hilden, Germany) at 63° C. in triplicates. The positive control contained 3,400 copies/reaction of synthetic HIV-1 RNA, the negative control contained no HIV-1 RNA. The fluorescence data were normalized to the initial value at 0 min (FIG. 12).

The detection method was also successfully applied in an RT-LAMP of human T-lymphotropic virus (HTLV-1) and tobacco mosaic virus (TMV) RNA. The performance and reaction conditions were identical to the already described RT-LAMP of HIV-1, but with sequence-specific primers for HTLV-1 and TMV. The fluorescence data were normalized to the initial value at 0 min (FIGS. 28 and 29).

### Example 17: Procedure According to Invention Using Non-Isothermal Amplification Reactions

In further examples of the invention, the detection method according to the invention can be used to detect DNA in a non-isothermal amplification reaction, such as PCR or PCDR. This involves modifying one or more primers in such a way that they represent a mediator probe. In a suitable reaction vessel, the sample and the required reagents are placed and the mixture incubated. During this process the fluorescence is detected in the reaction vessel. In the following, the execution example is described in detail using a PCDR:

The primers listed in Table 3 were used for real-time PCDR detection of mouse DNA. The PCDR primers for the amplification of G3PDH DNA were taken from (Ignatov et al. 2014) and partially modified. The mediator probe was created using the F3 primer by attaching a mediator binder region to the 5′ end of the primer, which can hybridize with a mediator. Mediator, F3 with mediator binding region and the detection molecule were created manually using VisualOMP. The primers as well as the synthetic oligonucleotides mediator, F3 with mediator binding sequence and detection molecule were synthesized by Biomers (biomers.net, Ulm, Germany). The mouse G3PDH DNA sequence to be amplified was taken from (Ignatov et al. 2014) and the G3PDH fragment was synthesized by Integrated DNA Technologies (IDT, Coralville, Iowa).

TABLE 3
Primer, mediator and detection molecule sequences for a real-time
PCDR for the detection of mice G3PDH DNA.

| | |
|---|---|
| F1 | GTGAAGGTCGGTGTGAACGGA |
| F2 | TTCTGCCGATGCCCATGT |
| F3 | GCACCTGCACCACCAACTG |
| R1 | GGTTTCTTACTCCTTGGAGGC |
| R2 | CAGATCCACGACGGACACATT |
| R3 | GAGCCCCGTTCAGCTCTG |
| Mediator | TAAAGCCATAGCCGTACTAGCTGCTCCAGTTCGGTCAGTG |
| F3 with | CACTGACCGAACTGGACTGGAGCAGCTAGTACGGCTATGGCTTTAGCATC |
| mediator | CTGCACCACCAACTG |
| binding | |
| sequence | |
| Detection | BMN-Q-535-CACCGGGCCAAGACGCGCCGGGG(dT-Atto- |
| molecule | 647N)GTGTTCACTGACCGAACTGGGAGCA |

PCDR was performed with SD Hotstart DNA Polymerase in 1×SD buffer (Bioron, Ludwigshafen, Germany). In addition, MgCl2 (Bioron, Ludwigshafen, Germany), final concentration 2.75 mM, and dNTPs (New England Biolabs, Frankfurt, Germany), final concentration 0.25 mM, were added to the buffer. The PCDR reaction consisted of 0.1 μM F3 and F3 each with mediator binding region, 0.2 μM R3, 0.1 μM F2 and R2, 0.05 μM F1 and R1, 0.05 μM mediator, 0.05 μM detection molecule, 200 U/ml SD Hotstart DNA Polymerase and 1×SD buffer. The reaction was carried out in a rotor gene 6000 (Corbett, Mortlake, Australia, now Qiagen, Hilden, Germany) according to the following protocol (Ignatov et al. 2014): Initial denaturation at 92° C. for 2 min, followed by 45 cycles at 92° C. (15 sec) and 66° C. (40 sec). The fluorescence data were normalized to the initial value at 0 cycles (FIG. 30). The positive control contained 100 pg G3PDH fragment per reaction, the negative control contained no template DNA.

### Example 18: Invented Mediator Probe that does not Act as Primer

In further examples, the detection according to the invention method can be used for the detection of DNA or RNA with increased specificity. A primer does not serve as a mediator probe, but a special probe is used which does not serve as a starting point for amplification. In the absence of the target molecule, the probe is closed. As soon as the target molecule is in the reaction mixture, the mediator probe binds to the target molecule or template molecule, whereby a primer can bind to the 3′ end of the now opened mediator probe. By processing with a suitable enzyme system, the attached primer can be extended, whereby the mediator is displaced by the mediator probe. The released mediator can be detected with the help of a specific detection molecule. The enzymatic amplification process may include, but is not limited to, isothermal processes (FIG. 13).

### Example 19: Use of Target Molecule-Specific Aptamers

In further versions, the detection according to the invention method for the detection of target molecules by target molecule-specific aptamers can be applied. Target molecule-specific aptamers, the sample to be investigated and detection molecules are placed in a suitable reaction vessel. The target molecule to be detected can be a protein or peptide, for example, but is not limited to it. An aptamer binds to the target molecule and changes its structure so that an aptamer-specific mediator probe and primer can attach after interaction. By processing with a suitable enzyme system, primers attached to the aptamer (FIG. 14: white marker in the aptamer) can be prolonged, resulting in amplification of the aptamer sequence outside the protein binding region. By binding a mediator probe to a linear amplification product, the probe is opened and further primers are used to displace the mediator from the mediator probe. The released mediator can be detected using a specific detection molecule or a suitable detection method. The enzymatic amplification process may include, but is not limited to, isothermal processes (FIG. 14).

### Example 20: Modified Mediator Probes, which have an Aptamer Region, a Mediator Binding Region and a Primer Binding Region

In preferred designs, the ingenious detection method can be used to detect target molecules using modified mediator probes, which have an aptamer region, a mediator binding region and a primer binding region. The target molecule to be detected can be a protein or peptide, for example, but is not limited to this. In the absence of the target molecule, the primer binds to the mediator probe and can be prolonged by processing with a suitable enzyme system, displacing the mediator from the mediator probe. The released mediator can trigger a detectable signal using a specific detection molecule or method. If the target molecule is present, the aptamer region of the mediator probe binds to the target molecule, whereby the primer attached to the primer binder region cannot be extended (FIG. 15). If the target molecule is present, a signal drop is detected in comparison to the absence of the target molecule. The enzymatic amplification process may include, but is not limited to, isothermal processes.

### Example 21: Use of an Aptamer Comprising a Protein Binder Region Flanked by Primer Binder Regions

In order to generate an exponential detection reaction, a mediator probe is used which consists of a primer with a mediator hybridization sequence at the 5′ end and a mediator hybridized to it. In addition, an aptamer is used, which is a protein binding region flanked by primer binding regions. In the presence of the target molecule, the aptamer binds to the target molecule. Primers that bind to the aptamer cannot be extended due to the binding to the target molecule. Consequently, the mediator is not released and, in the presence of the target molecule, a drop in signal compared to the absence of the target molecule is detected. In the absence of the target molecule, primers can bind to the aptamer and be prolonged, which leads to a signal increase through mediator release. The enzymatic amplification process may include, but is not limited to, isothermal methods (FIG. 16).

### Example 22: Immobilization of Detection Molecules

In other preferred versions of the detection method according to the invention, the detection molecules can be immobilized in a suitable reaction vessel on a solid phase. The sample and the required reagents are then added to the reaction vessel and the mixture incubated under the appropriate conditions. The sample may consist of DNA, RNA and/or peptides or proteins. If the target molecule is present, the mediator is displaced by the mediator probe and can diffuse in the reaction mixture to the immobilized detection molecule. The procedure includes but is not limited to isothermal amplification procedures (FIG. 17).

### Example 23: Use of Relaxometry

In further versions, the detection according to the invention method can be used in combination with magnetic relaxometry for the detection of target molecules. The detection molecules can be bound to magnetic particles and allow detection by magnetic relaxometry. In magnetic relaxometry, the magnetic particles are magnetized by a short magnetic pulse and the temporal degradation of the induced magnetic moment is detected. The hydrodynamic resistance of particles to which mediators bind and are extended via the detection molecules immobilized on the particles is greater, i.e. the hydrodynamic resistance of particles to which no mediators bind. Particles to which mediators bind and are extended therefore degrade their induced magnetic moment more slowly than particles to which no mediators bind. The relaxation times of the induced magnetic moments of the mentioned particles therefore differ from each other, whereby the release of mediators can be detected. By combining magnetic relaxometry with a melting curve analysis, different target molecules can be detected side by side in one sample. The procedure includes, but is not limited to, isothermal amplification procedures.

### Example 24: Use of Magnetic or Magnetizable Particles

In other designs, the detection according to the invention method can be used in combination with magnetic or magnetizable particles to detect target molecules (FIG. 31). The mediators are bound to magnetic or magnetizable nanoparticles. Several mediators can be bound to one particle at the same time. According to the invention, the mediators hybridize initially with primers. During amplification of the target or template molecule, the mediator is displaced by the primer and can then hybridize with a detection molecule immobilized on the solid phase. The binding between the released mediator and the detection molecule brings the nanoparticles to the surface of the solid phase, which enables a change in the magnetic properties to be detected. For the detection of a signal change on the surface of the solid phase, magnetic field sensors can be used, among other things, which, for example, but not exclusively, are based on galvano-magnetic, magneto-resistive, magneto-optical effects or on the Josephson effect. In order to prevent false positive signals due to the deposition of the non-released mediator/particle units on the solid phase, the non-released mediator/particle units can be separated from the solid phase by applying a weak magnetic field.

### Example 25: Electrochemical Detection with Detection Molecules Having a Hairpin Structure

In further versions, the detection according to the invention method can be used in combination with electrochemical detection to detect target molecules. In this version, the detection molecule is immobilized on an electrode, which simultaneously represents the solid phase. The released mediator can hybridize with the detection molecule in the mediator binding region and be extended by a polymerase. The mediator binding region may be located in different regions of the detection molecule. The detection molecule may have a hairpin structure and be marked with a redox molecule at the 5′ end. The extension of the mediator displaces the 5′ end of the detection molecule and opens the latter. Due to the displacement of the marked 5′ end, the distance between the redox molecule and the electrode surface increases, resulting in a detectable signal change. The procedure includes, but is not limited to, isothermal amplification procedures (FIG. 18).

### Example 26: Proof by Electrochemical Detection on a Solid Phase

In this preferred design, detection by electrochemical detection can take place on a solid phase. In this version, the detection molecule is immobilized on an electrode, which simultaneously represents the solid phase. The released mediator can hybridize with the detection molecule in the mediator binding region and be extended by a polymerase. After extension, redox molecules can intercalate into the dimer of detection molecule and extended mediator and generate an electrochemical signal that can be detected (FIG. 8). Signal generation can also take place without extension of the mediator according to FIG. 20. The mediator may be label-free and intercalating redox molecules may be used and/or the mediator may be labeled with one or more redox molecules. If the mediator is labeled with a redox molecule, the binding of the released mediator and, if necessary, subsequent extension of the mediator to the detection molecule leads to signal generation as described in FIGS. 21-23. In another version, the detection molecule is marked with one or more redox molecules. The binding of the released mediator and possibly subsequent extension of the mediator to the detection molecule leads to a signal change according to FIG. 24. It may be advantageous to release several mediators per target molecule and/or amplicon to obtain a stronger signal. The release of several mediators per target molecule can be achieved, for example, by attaching mediators to several different mediator probes.

In the following, electrochemical detection according to FIG. 21 will be discussed. The mediator is labeled with (a) redox molecule(s) and is released during amplification. In this version, the unlabeled detection molecule is immobilized on an electrode, which simultaneously represents the solid phase, and the released mediator can hybridize with the detection molecule in the mediator binder region. Due to the spatial proximity between the redox molecule on the mediator and the electrode surface, a signal change can be detected. Detection can take place in real time during the amplification reaction or as endpoint detection by transferring the amplification products to the electrode surface. The execution example is described in detail below using the electrochemical endpoint detection of the amplification products of a LAMP of E. coli:

The LAMP reaction was performed as described in example 15. However, for electrochemical detection mediator, LoopF with mediator binding sequence and detection molecule were adapted accordingly (Table 1). LoopF with mediator binding sequence and detection molecule were synthesized by Biomers (biomers.net, Ulm, Germany) and the mediator, which is modified with a methylene blue derivative (Atto MB2), by IBA Lifesciences (Göttingen, Germany).

TABLE 1
LoopF with mediator binding sequence, mediator and detection molecule
Sequences for the electrochemical detection of a LAMP of E. coli.

| | |
|---|---|
| mediator | Atto MB2-TCGTTCTGGGGCTCTACGACC |
| LoopF with mediator | GGTCGTAGAGAGCCCAGAACGATGCGCCATGTCCCGCT |
| binding sequence | |
| detection molecule | TTTTTTTTTTTTGGTCGTAGAGCCCAGAACGA |

After performing the LAMP of E. coli DNA as described in example 15, the reaction mix was transferred into a chamber with an electrode on which the detection molecules are immobilized. The electrochemical detection of the released mediators in the positive control reaction (60,000 copies of E. coli DNA) was performed by square wave voltammetry (FIG. 32). In the positive control, the mediators are displaced during the LAMP reaction and can hybridize to the detection molecule after transferring the reaction mix into the electrode chamber. Accordingly, the marked (here methylene blue) mediator accumulates on the electrode surface, which leads to the formation of a characteristic peak at −0.39 V in electrochemical analysis (here square wave voltammetry). In contrast, the absence of a peak in negative control (NTC) indicates that there was no significant release of mediators. The application of the detection method according to the invention in combination with the electrochemical detection proves to be particularly advantageous here, since after the amplification reaction there is no need to carry out further modifications or reaction steps.

### Example 27: Parallel Detection of DNA, RNA, Peptides and/or Proteins

In a preferred version of the method according to the invention, DNA, RNA and peptides or proteins or another combination of the mentioned substance classes are detected in parallel by the described methods in one approach. The procedure includes, but is not limited to, isothermal amplification procedures.

### REFERENCES

Das, Jagotamoy; Cederquist, Kristin B.; Zaragoza, Alexandre A.; Lee, Paul E.; Sargent, Edward H.; Kelley, Shana O. (2012): An ultrasensitive universal detector based on neutralizer displacement. In: Nature chemistry 4 (8), S. 642-648. DOI: 10.1038/nchem.1367.
Curtis, Kelly A.; Rudolph, Donna L.; Owen, S. Michele (2008): Rapid detection of HIV-1 by reverse-transcription, loop-mediated isothermal amplification (RT-LAMP). In: Journal of virological methods 151 (2), S. 264-270. DOI: 10.1016/j.jviromet.2008.04.011.
Faltin, Bernd; Wadle, Simon; Roth, Gunter; Zengerle, Roland; Stetten, Felix von (2012): Mediator probe PCR: a novel approach for detection of real-time PCR based on label-free primary probes and standardized secondary universal fluorogenic reporters. In: Clinical chemistry 58 (11), S. 1546-1556. DOI: 10.1373/clinchem.2012.186734.
G. J. Nuovo; R. J. Hohman; G. A. Nardone; I. A. Nazarenko (1999): In Situ Amplification Using Universal Energy Transfer-labeled Primers. In: The Journal of Histochemistry & Cytochemistry (47), S. 273-279.
Ignatov, Konstantin B.; Barsova, Ekaterina V.; Fradkov, Arkady F.; Blagodatskikh, Konstantin A.; Kramarova, Tatiana V.; Kramarov, Vladimir M. (2014): A strong strand displacement activity of thermostable DNA polymerase markedly improves the results of DNA amplification. In: BioTechniques 57 (2), S. 81-87. DOI: 10.2144/000114198.
Li, Xiaomin; Huang, Yong; Guan, Yuan; Zhao, Meiping; Li, Yuanzong (2006): Universal molecular beacon-based tracer system for real-time polymerase chain reaction. In: Analytical chemistry 78 (22), S. 7886-7890. DOI: 10.1021/ac061518.
Li, Xiaomin; Huang, Yong; Song, Chen; Zhao, Meiping; Li, Yuanzong (2007): Several concerns about the primer design in the universal molecular beacon real-time PCR assay and its application in HBV DNA detection. In: Analytical and bioanalytical chemistry 388 (4), S. 979-985. DOI: 10.1007/s00216-007-1281-4.
Linardy, Evelyn M.; Erskine, Simon M.; Lima, Nicole E.; Lonergan, Tina; Mokany, Elisa; Todd, Alison V. (2016): EzyAmp signal amplification cascade enables isothermal detection of nucleic acid and protein targets. In: Biosensors & bioelectronics 75, S. 59-66. DOI: 10.1016/j.bios.2015.08.021.
Tanner, Nathan A.; Zhang, Yinhua; Evans, Thomas C., JR (2012): Simultaneous multiple target detection in real-time loop-mediated isothermal amplification. In: BioTechniques 53 (2), S. 81-89. DOI: 10.2144/0000113902.
Yang, Litao; Liang, Wanqi; Jiang, Lingxi; Li, Wenquan; Cao, Wei; Wilson, Zoe A.; Zhang, Dabing (2008): A novel universal real-time PCR system using the attached universal duplex probes for quantitative analysis of nucleic acids. In: BMC molecular biology 9, S. 54. DOI: 10.1186/1471-2199-9-54.

1. Mediator probe for the detection of at least one target molecule, wherein the mediator probe comprises at least two oligonucleotides, characterized in that
a) a first oligonucleotide comprises a probe region and a mediator binding region, wherein
i. the probe region has an affinity to a target molecule and/or template molecule, and
ii. the mediator binding region has an affinity for at least one mediator, and
b) at least one further oligonucleotide is a mediator which is
i. is bound via the mediator binding region to the first oligonucleotide of the mediator probe, and
ii. has an affinity for at least one detection molecule, wherein the mediator triggers a detectable signal after release from the first oligonucleotide of the mediator probe by interaction with the detection molecule.

2. A mediator probe according to claim 1, characterized in that the first oligonucleotide of mediator probe and/or of mediator does not comprise a marker for signal generation.

3. Mediator probe according to claim 1 or 2, characterized in that the first oligonucleotide of mediator probe and/or the mediator contains one or more markers for signal generation, preferably a fluorescent molecule, a redox molecule, a luminescent molecule or another signal generating unit.

4. System comprising at least one mediator probe according to one of claims 1 to 3 and at least one detection molecule, characterized in that the at least one detection molecule comprises one or more oligonucleotides and comprises at least one first region which interacts with at least one mediator, and
a) a second region comprising a fluorescence acceptor or a fluorescence donor and/or a chemical group for binding to a solid phase and/or a chemical protecting group and/or redox modifications and/or luminescence modifications, and/or
b) a third region comprising a fluorescence donor or a fluorescence acceptor and/or a chemical group for binding to a solid phase and/or a chemical protecting group and/or redox modifications and/or luminescence modifications,
or
c) at least one fourth region which interacts with at least one first probe which has a fluorescence donor and/or a fluorescence acceptor, and/or
d) at least one fifth region interacting with at least one second probe comprising a fluorescence donor and/or a fluorescence acceptor.

5. System according to the previous claim, characterized in that the detection molecule has a hairpin structure.

6. A method of detecting at least one target molecule comprising the following steps:
a) Providing at least one mediator probe according to claims 1 to 3 and/or of a system according to claim 4 or 5,
b) Binding of the probe region of the first oligonucleotide of the at least one mediator probe to a sequence of the template molecule and/or of the target molecule,
c) Amplifying the first oligonucleotide of the at least one mediator probe and/or of the template molecule and/or of the target molecule,
d) Releasing at least one mediator by at least one auxiliary molecule,
e) Optional binding of the at least one released mediator to the at least one detection molecule, and
f) Detecting a signal change.

7. Method according to the preceding claim, characterized in that at least one mediator binds to the first region of the detection molecule and is enzymatically extended by at least one auxiliary molecule, the auxiliary molecule preferably binding to the 3′ terminus of the bound mediator, whereby a physically or chemically measurable change in the detection molecule occurs.

8. Process according to one of claim 6 or 7, characterized in that the 3′ terminus of the first oligonucleotide of the mediator probe is enzymatically extended by an auxiliary molecule after binding of the probe region of the first oligonucleotide of the mediator probe to a sequence of the template molecule and/or of the target molecule.

9. A process according to any of claims 6 to 8, characterized in that the amplification of the first oligonucleotide of the mediator probe and/or the template molecule and/or target molecule occurs via by an isothermal or non-isothermal amplification method.

10. Method according to one of claims 6 to 9, characterized in that the detection molecule has at least one fluorescence or luminescence modification and, after the reaction with the at least one mediator, the fluorescence or luminescence modifications are cleaved from the detection molecule by means of an auxiliary molecule and/or the 5′ terminus of the hairpin structure of the detection molecule is removed and/or the hairpin structure is unfolded and a change in the fluorescence signal or luminescence signal is detected on the detection molecule.

11. Method according to one of claims 6 to 10, characterized in that several mediators are used per mediator probe and/or several mediator probes and/or several detection molecules per target molecule.

12. A method according to any of claims 6 to 11, characterized in that the at least one auxiliary molecule has a DNA strand separating effect and/or a polymerizing effect, wherein the auxiliary molecule is preferably a strand displacement polymerase.

13. A method according to any of claims 6 to 12, characterized in that the target molecule and/or the template molecule is a biomolecule selected from the group consisting of DNA, RNA, peptide, protein, aptamer and/or a combination thereof.

14. A process according to any of claims 6 to 13, characterized in that the auxiliary molecule is selected from the group consisting of polymerases, catalysts, proteins, nucleic acids, natural substances, enzymes, enzyme systems, cell lysates, cell components, derivatives derived from cell components and/or synthetic molecules.

15. A method according to any of claims 6 to 14, characterized in that a measurable change in fluorescence, phosphorescence, luminescence, mass, absorption, scattering of light, electrical conductivity, enzymatic activity and/or affinity, electrochemical potential or signal, refractive index, triggering of surface plasmons, magnetic relaxation, magnetic property, impedance or capacitance occurs by direct or indirect interaction between immobilized or non-immobilized detection molecule and at least one mediator.

16. A method according to any of claims 6 to 15, characterized in that the release of the at least one mediator is detected by amplification of the at least one mediator by means of an isothermal or non-isothermal amplification method.

17. Method according to one of claims 6 to 16, characterized in that the at least one released mediator is detected by sequencing.

18. Process according to one of claims 6 to 17, characterized in that the at least one released mediator binds to the detection molecule by hybridization, is optionally extended by an auxiliary molecule after binding to the detection molecule, and then a melting curve analysis is carried out.
